(19) United States
(12) Patent Application Publication
(10) Pub. No.: US 2021/0275506 A1
(43) Pub. Date: Sep. 9, 2021

(54) COMPOSITIONS AND METHODS OF TREATMENT

(71) Applicant: Blueberry Therapeutics Limited, Macclesfield Cheshire (GB)

(72) Inventors: Christine Ridden, Macclesfield Cheshire (GB); John Ridden, Macclesfield Cheshire (GB); David Cook, Macclesfield Cheshire (GB)

(21) Appl. No.: 17/257,423

(22) PCT Filed: Jul. 3, 2019
(86) PCT No.: PCT/GB2019/051888

§ 371 Date: Dec. 31, 2020

(30) Foreign Application Priority Data
Jul. 3, 2018 (GB) 1810923.1

(51) Int. Cl. A61K 31/436; A61K 9/16; A61K 31/192; A61K 38/13; A61K 31/196; A61P 17/00; A61P 29/00

(52) U.S. Cl. CPC A61K 31/436; A61K 9/1641; A61K 31/192; A61P 29/00; A61K 31/196; A61P 17/00; A61K 38/13

(57) ABSTRACT

The present invention relates to compositions for use in the treatment of a dermatological condition, comprising a polymer capable of forming nanoparticles an anti-inflammatory and/or analgesic agent. The composition is particularly suited to the treatment of acne and/or atopic dermatitis. The invention also relates to novel uses of polyhexamethylene biguanide.

### TECHNICAL FIELD OF THE INVENTION

The present invention relates to a composition for the topical treatment of dermatological conditions, such as acne, atopic dermatitis and other related skin conditions.

### BACKGROUND TO THE INVENTION

Acne disorder is most common in people aged 15-25 years. The symptoms of acne include “blackheads”, “whiteheads” and inflammed spots usually on the face or back. These are formed when excess sebaceous secretion in a hair follicle causes it to become blocked. Spots may then become infected by bacteria leading to them to develop into inflamed cysts and pustules. Acne symptoms range from mild, only a few spots, to severe cases with many deep, inflamed and infected spots that may result in permanent scarring.

Acne is a disease of the sebaceous hair follicles, often called pores. At the base of each hair follicle is a gland called the sebaceous gland, which produces sebum. Sebum is an oily substance that keeps the skin moist and pliable, which under normal circumstances travels along the hair follicle to the surface of the skin. A blemish begins approximately 2-3 weeks before it appears on the skin's surface. As the skin renews itself, the old cells die and slough off. When cells are shed unevenly and clump together with the sebum it forms a plug. Sebum which normally drains to the surface gets blocked and bacteria begin to grow. The rapid growth of the bacteria in combination with the accumulated sebum cause the follicle to enlarge and result in a mild form of acne called comedones, which are non-inflammatory. Both whiteheads and blackheads start out as a “microcomedone” and then become skin blemishes called comedones, either a whitehead or a blackhead. Acne is trapped sebum and bacteria (Propionibacterium acnes) growing in a plugged follicle. Sebaceous glands are most numerous on the face, chest, back, neck and scalp; consequently, these are the most common sites of acne. The most common factors that cause acne are hormones, increased sebum production, bacteria (Propionibacterium Acnes), and changes inside of the hair follicle. Acne may progress to an inflammatory type of acne lesions that are red in color called papules, pustules and nodules.

There are many types of acne, ranging in severity from mild to severely disfiguring. Acne vulgaris is the most common form of acne which includes several types of pimples. These acne lesions include blackheads, whiteheads, papules, pustules, nodules and cysts.

Mild to moderate acne vulgaris is characterized by whiteheads, blackheads, papules, and pustules. A whitehead is formed when a pore is completely blocked, trapping sebum, bacteria, and dead skin cells below the skin surface causing a white appearance on the surface. Whiteheads are normally quicker in life cycle than blackheads. A blackhead is formed when a pore is only partially blocked, allowing some of the trapped sebum, bacteria, and dead skin cells to drain to the surface slowly. The black colour is due to a reaction of the skin's own pigment, melanin, reacting with the oxygen in the air. A blackhead tends to be a stable structure. Blackheads can often take a long time to clear because the contents very slowly drain to the surface. Papules are small, red, tender bumps with no head. Papules are the earliest stage in the development of what are normally considered the typical “pimple”. Papules are an intermediate in the progression of acne between the non-inflammatory and inflammatory stages. Pustules are similar to whiteheads, but are inflamed, and appear as a red circle with a white or yellow center.

Severe acne vulgaris is characterized by nodules and cysts. Nodular acne consists of acne spots which are much larger, can be quite painful, and can sometimes last for months. Nodules are large, hard bumps under the skin's surface. Scarring is common with nodules. An acne cyst can appear similar to a nodule, but is pus-filled, and can been described as having a diameter of 5 mm or more across. They can be painful and scarring is common with cystic acne.

Acne rosacea can look similar to the aforementioned acne vulgaris, and the two types of acne are sometimes confused for one another. Rosacea affects millions of people, most of whom are over the age of 30. It appears as a red rash which is normally confined to the cheeks, nose, forehead and chin. The redness is often accompanied by bumps, pimples, and skin blemishes. Blood vessels may also become more visible on the skin. Blackheads are not a part of rosacea. It is more prevalent in women, but often more severe when found in men. Left untreated, it can cause swelling of the nose and the growth of excess tissue, a condition called rhinophyma.

Acne conglobata is the most severe form of acne vulgaris and is more common in males. It is characterized by numerous large lesions, which are sometimes interconnected, along with widespread blackheads. It can cause severe, irrevocable damage to the skin, and disfiguring scarring. It is found on the face, chest, back, buttocks, upper arms, and thighs. The age of onset for acne conglobata is usually between 18 and 30 years, and the condition can stay active for many years.

Acne fulminans is an abrupt onset of acne conglobata which normally afflicts young men. Symptoms of severe nodulocystic, often ulcerating acne are apparent. As with acne conglobata, extreme, disfiguring scarring is common. Acne fulminans is unique in that it also includes a fever and aching of the joints.

Gram-negative folliculitis is a bacterial infection characterized by pustules and cysts, possibly occurring as a complication resulting from a long term antibiotic treatment of acne vulgaris. It is a rare condition, and prevalence in males versus females is unknown.

Pyoderma Faciale is severe facial acne affects only females, usually between the ages of 20 to 40 years old, and is characterized by painful large nodules, pustules and sores which may leave scarring. It begins abruptly, and may occur on the skin of a woman who has never had acne before. It is confined to the face, and usually does not last longer than one year, but can wreak havoc in a very short time.

Teenage (adolescent) acne: Most cases of acne that require treatment occur in individuals 9 to 19 years of age. Boys and girls are equally affected but the condition is usually more severe in boys. No ethnic groups are predisposed to acne, but certain cosmetic practices, such as the use of oily grooming agents, can lead to a specific pattern of lesions. Internal factors that may cause adolescents acne include endogenous hormones (androgens, progesterone) and specific drugs (oral contraceptives, isoniazid, phenytoin, corticosteroids, lithium-containing compounds). External factors include skin bacteria, especially Propionibacterium acnes; industrial chemicals (petroleum, animal and vegetable oils); oil- or wax-containing cosmetics; greasy sunscreen or suntan preparations; and local pressure from objects such as headbands, shoulder pads, or helmets. Excessive perspiration and emotional stress can also aggravate acne. Androgens (e.g., testosterone) will increase the size of sebaceous glands and, in people prone to acne, increase the production of sebum. In women, fluctuations in estrogen during the menstrual cycle change the sensitivity of sebaceous glands to androgens. During puberty, the skin cells lining the follicle shed more quickly, mix with the increased levels of sebum and increase the likelihood of the pores becoming clogged.

Adult acne: Twenty percent (20%) of the adult population is afflicted by adult acne, most of them women. Modern-day job related stress, pollution, poor nutrition and bad cosmetics are among the major contributing factors. A common case of adult acne consists of blackheads and whiteheads, while others developed inflamed acne papules or pustules. Approximately 85% of people worldwide suffer from acne at some point in their lives, leading to more than 103 million affected by acne at any given moment. Approximately 17 million people in the U.S. have acne resulting in approximately 5.5 million visits to the physicians each year.

Acne vulgaris occurs in up to 95% of the population in westernized societies; acne vulgaris is a nearly universal skin disease afflicting 79% to 95% of the adolescent population. In men and women older than 25 years, 40% to 54% have some degree of facial acne, and clinical facial acne persists into middle age in 12% of women and 3% of men. (Cordain L, Lindeberg S, Hurtado M, Hill K, Eaton S B, Brand-Miller J. Acne vulgaris: a disease of Western civilization. Arch Dermatol 2002 December; 138(12):1584-90).

Current medications include a variety of topical and systemic medications such as antibiotics, anti-infectives, anti-inflammatories, hormone therapies, keratolytics, and retinoids. The over the counter medication include benzoyl peroxide, salicylic acid, sulfur, and resorcinol.

Benzoyl peroxide medication is very effective for killing acne-causing bacteria. Benzoyl peroxide first saw use in the 1930's, and remains a mainstay of acne treatment because it has proven itself to work well. To this day, benzoyl peroxide actually kills Propionibacterium acnes (P. Acnes) better than any other medication on the market, prescription or otherwise. Benzoyl peroxide is available in non-prescription concentrations of 2.5%, 5% and 10%. Many anti-acne agents, including benzoyl peroxide, have a high flux into the skin. While this penetration is advantageous during the initial application, the rapid subsequent diffusion in the skin means that the anti-acne agent will diffuse away from the acne lesion to be treated (a phenomenon known as “outflow”). In turn, this means that the efficacy of the anti-acne agent is lessened, because of the relatively short dwell time of the anti-acne agent in the area of the acne lesion. Thus, it would be advantageous to have a method and a kit that would decrease the outflow of the anti-acne agent from the area of the acne lesion.

Currently Acne is treated in a number of ways. From simple antibacterial skin wash through to oral antibiotics and retinoids. In addition, to these 2 strong treatment regimes acne can also be treated with topical antibiotics, Salicylic acid based medicines, topical retinoids, in woman oral contraceptives and azelaic acid. Of these treatments the most effective are GP prescribed oral antibiotics. Although antibiotic resistance is a growing concern. Retinoids have serious safety concerns in the youth especially with regard to depression, headaches, dry skin etc.

An object of the present invention is to address one or more of the above problems associated with acne and/or atopic dermatitis treatments. It is also an object of the present invention to provide an acne and/or atopic dermatitis treatment. It is additionally an object of the present invention to provide a treatment which allows for better penetration or delivery of anti-inflammatory treatments. It is also an object of the present invention to provide an effective face wash suitable for regular use which will also reduce inflammation associated with acne and/or atopic dermatitis.

### SUMMARY OF THE INVENTION

In accordance with a first aspect of the present invention, there is provided a composition for use in the treatment of dermatological condition, comprising a polymer capable of forming nanoparticles and an anti-inflammatory and/or analgesic agent.

Related to the first aspect of the present invention, there is provided a composition for the treatment of dermatological condition, comprising a polymer capable of forming nanoparticles and an anti-inflammatory and/or analgesic agent.

Further related to the first aspect of the present invention, there is provided the use of a composition comprising a polymer capable of forming nanoparticles and an anti-inflammatory and/or analgesic agent, in the manufacture or preparation of a medicament for the treatment of a dermatological condition.

The polymer comprises a linear and/or branched or cyclic polymonoguanide/polyguanidine, polybiguanide, analogue or derivative thereof.

By forming nanoparticles from polymers and an anti-inflammatory and/or analgesic agent, the inventors have advantageously found that it is possible to enhance the delivery of the anti-inflammatory and/or analgesic agent into and through the stratum corneum.

It is preferred that the polymer comprises a linear and/or branched or cyclic polymonoguanide/polyguanidine, polybiguanide, analogue or derivative thereof. The linear and/or branched or cyclic polymonoguanide/polyguanidine, polybiguanide, analogue or derivative thereof may be according to the following formula 1a or formula 1b, with examples provided in tables A and B below:

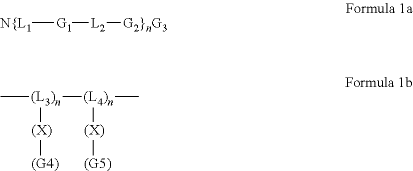

wherein:

“n”, refers to number of repeating units in the polymer, and n can vary from 2 to 1000, for example from 2 or 5 to 10, 20, 30, 40, 50, 60, 70, 80, 90, 100, 150, 200, 250, 300, 350, 400, 450, 500, 600, 700, 800 or 900;

G₁ and G₂ independently represent a cationic group comprising biguanide or guanidine, wherein L₁ and L₂ are directly joined to a Nitrogen atom of the guanide. Thus, the biguanide or guanidine groups are integral to the polymer backbone. The biguanide or guanidine groups are not side chain moieties in formula 1a.

Example of cationic groups:

### Biguanide

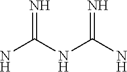

### (as in PHMB) or

guanidine

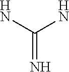

### (as in PHMG)

In the present invention, L₁ and L₂ are the linking groups between the G₁ and G₂ cationic groups in the polymer. L₁ and L₂ can independently represent an aliphatic group containing C₁-C₁₄₀ carbon atoms, for example an alkyl group such as methylene, ethylene, propylene, C₄, C₅, C₆, C₇, C₈, C₉ or C₁₀; C₁-C₁₀, -C₂₀, —C₃₀, -C₄₀, -C₅₀ -C₆₀, -C₇₀, -C₈₀, -C₉₀, -C₁₀₀, -C₁₁₀, -C₁₂₀, -C₁₃₀ or -C₁₄₀, alkyl; or L₁ and L₂ can (independently) be C₁-C₁₄₀ (for example C₁, C₂, C₃, C₄, C₅, C₆, C₇, C₈, C₉ or C₁₀; C₁-C₁₀, -C₂₀, -C₃₀, -C₄₀, -C₅₀-C₆₀, -C₇₀, -C₈₀, -C₉₀, -C₁₀₀, -C₁₁₀, -C₁₂₀, -C₁₃₀ or -C₁₄₀), cycloaliphatic, heterocyclic, aromatic, aryl, alkylaryl, arylalkyl, oxyalkylene radicals, or L₁ and L₂ can (independently) be a polyalkylene radical optionally interrupted by one or more, preferably one, oxygen, nitrogen or sulphur atoms, functional groups as well as saturated or unsaturated cyclic moiety. Examples of suitable L₁ and L₂ are groups are listed in table A.

L₁, L₂, G₁ and G₂ may have been modified using aliphatic, cycloaliphatic, heterocyclic, aryl, alkaryl, and oxyalkylene radicals.

N and G₃ are preferably end groups. Typically the polymers of use in the invention have terminal amino (N) and cyanoguanidine (G₃) or guanidine (G₃) end groups. Such end groups may be modified (for example with 1,6-diaminohexane, 1,6 di(cyanoguanidino)hexane, 1,6-diguanidinohexane, 4-guanidinobutyric acid) by linkage to aliphatic, cycloaliphatic heterocyclic, heterocyclic, aryl, alkylaryl, arylalkyl, oxyalkylene radicals. In addition, end groups may be modified by linkage to receptor ligands, dextrans, cyclodextrins, fatty acids or fatty acid derivatives, cholesterol or cholesterol derivatives or polyethylene glycol (PEG). Optionally, the polymer can end with guanidine or biguanide or cyanoamine or amine or cyanoguanidine at N and G₃ positions or cyanoamine at N and cyanoguanidine at G₃ position or guanidine at N and Cyanoguanide at G₃ positions or L1 amine at G₃ and cyanoguanidine at N. G3 can be L₁-amine, L₂-cyanoguanidine or L₂-guanidine. Depending on the number of polymerization (n) or polymer chain breakage and side reactions during synthesis, heterogeneous mixture of end groups can arise as described above as an example. Thus, the N and G3 groups can be interchanged/present as a heterogeneous mixture, as noted above. Alternatively N and G₃ may be absent and the polymer may be cyclic, in which case the respective terminal L₁ and G₂ groups are linked directly to one another.

In formula 1 b, X can be either present or absent. L₃, L₄ and X are as noted above for “L₁ or L₂”. In Thus, L₃ and L₄ and X are the linking groups between the G₄ and G₅ cationic groups in the polymer. L₃ and L₄ and X can independently represent an aliphatic group containing C₁-C₁₄₀ carbon atoms, for example an alkyl group such as methylene, ethylene, propylene, C₄, C₅, C₆, C₇, C₈, C₉ or C₁₀; C₁-C₁₀, -C₂₀, -C₃₀, -C₄₀, -C₅₀-C₆₀, -C₇₀, -C₈₀, -C₉₀, -C₁₀₀, -C₁₁₀, -C₁₂₀, -C₁₃₀ or -C₁₄₀, alkyl; or L₃ and L₄ and X can independently be C₁-C₁₄₀ (for example C₁, C₂, C₃, C₄, C₅, C₆, C₇, C₈, C₉ or C₁₀; C₁-C₁₀, -C₂₀, -C₃₀, —C₄₀, -C₅₀ -C₆₀, -C₇₀, -C₈₀, -C₉₀, -C₁₀₀, -C₁₁₀, -C₁₂₀, -C₁₃₀ or -C₁₄₀), cycloaliphatic, heterocyclic, aromatic, aryl, alkylaryl, arylalkyl, oxyalkylene radicals, or L₃ and L₄ and X can independently be a polyalkylene radical optionally interrupted by one or more, preferably one, oxygen, nitrogen or sulphur atoms, functional groups as well as saturated or unsaturated cyclic moiety. Examples of suitable L₃ and L₄ and X are groups are listed in table B.

“G₄” and “G₅” are cationic moieties and can be same or different. At least one of them is a biguanidine moiety or carbamoylguanidine, and the other moiety may be as above (biguanidine or carbamoylguanidine) or amine. For the avoidance of doubt, in formula 1b, cationic moiety G₄ and G₅ do not contain only single guanidine groups. For example, G₄ and G₅ typically do not contain single guanidine groups. Examples of such compounds are polyallylbiguanide, poly(allylbiguanidnio-co-allylamine), poly(allylcarbamoylguanidino-co-allylamine), polyvinylbiguanide, as listed in table B.

Example of polyallylbiguanide is as shown below:

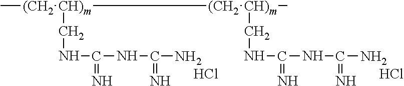

In case of polyallylbigunidine L₃ and L₄ are identical, G₄ and G5 are similar, thus polyallylbiguanide can be simplified as below.

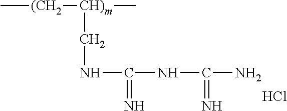

Example of poly(allylcarbamoylguanidnio-co-allylamine) is as shown below

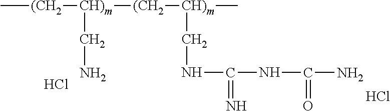

The polymers for use in the invention will generally have counter ions associated with them. Suitable counter ions include but are not limited to the following: halide (for example chloride), phosphate, lactate, phosphonate, sulfonate, amino carboxylate, carboxylate, hydroxy carboxylate, organophosphate, organophosphonate, organosulfornate and organosuflate.

Polymers for use in the invention can be either heterogeneous mixtures of polymers of different “n” number or homogenous fractions comprising specified “n” numbers purified by standard purification methods. As indicated above the polymers may also be cyclic and in addition may be branched.

Preferred numbers for “n” include 2-250, 2-100, 2-80 and 2-50.

TABLE A
Examples of polymer analogues arising from formula 1a.

| Name | L₁ | G₁ | L₂ | G₂ |
|---|---|---|---|---|
| Polyhexamethylene biguanide (PHMB) | (CH₂)₆ | Biguanide | (CH₂)₆ | Biguanide |
| Polyethylene biguanide (PEB) | (CH₂)₂ | Biguanide | (CH₂)₂ | Biguanide |
| Polyethylenetetramethylene biguanide | (CH₂)₂ | Biguanide | (CH₂)₄ | Biguanide |
| Polyethylene hexamethylene biguanide | (CH₂)₂ | Biguanide | (CH₂)₆ | Biguanide |
| (PEHMB) | | | | |
| Polypropylene biguanide, | (CH₂)₃ | Biguanide | (CH₂)₃ | Biguanide |
| Polyaminopropyl biguanide (PAPB) | | | | |
| Poly-[2-(2-ethoxy)- ethoxyethyl]- | (CH₂CH₂OCH₂CH₂OCH₂CH₂) | Biguanide | (CH₂CH₂OCH₂CH₂OCH₂CH₂) | Biguanide |
| biguanide- chloride] | | | | |
| (PEEG) | | | | |
| Polypropylenehexamethylene biguanide | (CH₂)₃ | Biguanide | (CH₂)₆ | Biguanide |
| Polyethyleneoctamethylene biguanide | (CH₂)₂ | Biguanide | (CH₂)₈ | Biguanide |
| Polyethylenedecamethylene biguanide | (CH₂)₂ | Biguanide | (CH₂)₁₀ | Biguanide |
| Polyethylenedodecamethylene | (CH₂)₂ | Biguanide | (CH₂)₁₂ | Biguanide |
| biguanide | | | | |
| Polytetramethylenehexamethylene | (CH₂)₄ | Biguanide | (CH₂)₆ | Biguanide |
| biguanide | | | | |
| Polytetramethylenebiguanide | (CH₂)₄ | Biguanide | (CH₂)₄ | Biguanide |
| Polypropyleneoctamethylene biguanide | (CH₂)₃ | Biguanide | (CH₂)₈ | Biguanide |
| Polytetramethyleneoctamethylene | (CH₂)₄ | Biguanide | (CH₂)₈ | Biguanide |
| Biguanide | | | | |
| Polyhexamethylene diethylenetriamine | (CH₂)₆ | Biguanide | CH₂—CH₂—NH—CH₂—CH₂ | Biguanide |
| biguanide | | | | |
| Polyhexamethylene guanide (PHMG) | (CH₂)₆ | guanidine | (CH₂)₆ | guanidine |
| Polyethylene guanide | (CH₂)₂ | guanidine | (CH₂)₂ | guanidine |
| Polyethylenetetramethylene guanide | (CH₂)₂ | guanidine | (CH₂)₄ | guanidine |
| Polyethylene hexamethylene guanide | (CH₂)₂ | guanidine | (CH₂)₆ | guanidine |
| Polypropylene guanide, | (CH₂)₃ | guanidine | (CH₂)₃ | guanidine |
| Polyaminopropyl guanide (PAPB) | | | | |
| Poly-[2-(2-ethoxy)- ethoxyethyl]- | (CH₂CH₂OCH₂CH₂OCH₂CH₂) | guanidine | (CH₂CH₂OCH₂CH₂OCH₂CH₂) | guanidine |
| guanide | | | | |
| Polypropylenehexamethylene guanide | (CH₂)₃ | guanidine | (CH₂)₆ | guanidine |
| Polyethyleneoctamethylene guanide | (CH₂)₂ | guanidine | (CH₂)₈ | guanidine |
| Polyethylenedecamethylene guanide | (CH₂)₂ | guanidine | (CH₂)₁₀ | guanidine |
| Polyethylenedodecamethylene guanide | (CH₂)₂ | guanidine | (CH₂)₁₂ | guanidine |
| Polytetramethylenehexamethylene | (CH₂)₄ | guanidine | (CH₂)₆ | guanidine |
| guanide | | | | |
| Polypropyleneoctamethylene guanide | (CH₂)₃ | guanidine | (CH₂)₈ | guanidine |
| Polytetramethylene guanide | (CH₂)₄ | guanidine | (CH₂)₄ | guanidine |
| Polyhexamethylene diethylenetriamine | (CH₂)₆ | guanidine | CH₂—CH₂—NH—CH₂—CH₂ | guanidine |
| guanide | | | | |

TABLE B
Examples of polymer analogues arising from formula 1b.

| Polymer | CAS Number |
|---|---|
| Polyhexamethylene biguanide hydrochloride | 27083-27-8 |
| (PHMB) | 32289-58-0 |
| Polyhexamethylene guanidine hydrochloride | 57028-96-3 |
| (PHMG) | |
| Poly-[2-(2-ethoxy)- ethoxyethyl]- | 374572-91-5 |
| guanidinium-chloride] (PEEG) | |

| Name | L₃ | G₄ | L₄ | G₅ | x |
|---|---|---|---|---|---|
| Polyallylbiguanide | (CH₂—CH) | Biguanide | (CH₂—CH) | Biguanide | CH₂ |
| poly(allylbiguanidnio-co- | (CH₂—CH) | amine | (CH₂—CH) | biguanide | CH₂ |
| allylamine) | | | | | |
| poly(allylcarbamoylguanidino- | (CH₂—CH) | amine | (CH₂—CH) | Carbamoyl | CH₂ |
| co-allylamine) | | | | guanidine | |
| polyvinylbiguanide | (CH₂—CH) | Biguanide | (CH₂—CH) | biguanide | absent |

CAS numbers for example compounds arising from formula 1a

The polymer used in the method of the invention may comprise linear, branched or dendrimeric molecules. The polymer may comprise a combination of linear, branched or dendrimeric molecules. The polymer may comprise one or any combination of molecules of Formula 1a or Formula 1 b, for example as described above.

For example, the polymer can comprise one or more of polyhexamethylene biguanide (PHMB), polyhexamethylene monoguanide (PHMG), polyethylene biguanide (PEB), polytetramethylene biguanide (PTMB) or polyethylene hexamethylene biguanide (PEHMB). Some examples are listed in table A and B.

Thus, the polymer may comprise homogeneous or heterogeneous mixtures of one or more of polyhexamethylene biguanide (PHMB), polyhexamethylene monoguanide (PHMG), polyethylene biguanide (PEB), polytetramethylene biguanide (PTMB), polyethylene hexamethylene biguanide (PEHMB), polymethylene biguanides (PMB), poly(allylbiguanidnio-co-allylamine), poly(N-vinylbiguanide), polyallybiguanide.

Most preferred the polymer comprises polyhexamethylene biguanide (PHMB).

In one embodiment, the anti-inflammatory and/or analgesic agent comprises the same active pharmaceutical ingredient. It will be apparent to the skilled addressee that certain anti-inflammatory agents have been shown to also have analgesic properties. In other embodiments, the composition comprises a separate anti-inflammatory and a separate analgesic agent.

The anti-inflammatory agent may comprise a number of different types of anti-inflammatory agents, including steroidal anti-inflammatory agents (SAID) and non-steroidal anti-inflammatory agents. In certain embodiments, it is preferred that the anti-inflammatory agent comprises a non-steroidal anti-inflammatory (NSAID) agent. Such a NSAID may be selected from one or more of the following: Aspirin (Anacin, Ascriptin, Bayer, Bufferin, Ecotrin, Excedrin); Choline and magnesium salicylates (CMT, Tricosal, Trilisate); Choline salicylate (Arthropan); Celecoxib (Celebrex); Diclofenac potassium (Cataflam); Diclofenac sodium (Voltaren, Voltaren XR); Diclofenac sodium with misoprostol (Arthrotec); Diflunisal (Dolobid); Etodolac (Lodine, Lodine XL); Fenoprofen calcium (Nalfon); Flurbiprofen (Ansaid); Ibuprofen (Advil, Motrin, Motrin IB, Nuprin); Indomethacin (Indocin, Indocin SR); Ketoprofen (Actron, Orudis, Orudis KT, Oruvail); Magnesium salicylate (Arthritab, Bayer Select, Doan's Pills, Magan, Mobidin, Mobogesic); Meclofenamate sodium (Meclomen); Mefenamic acid (Ponstel); Meloxicam (Mobic); Nabumetone (Relafen); Naproxen (Naprosyn, Naprelan); Naproxen sodium (Aleve, Anaprox); Oxaprozin (Daypro); Piroxicam (Feldene); Rofecoxib (Vioxx) Salsalate (Amigesic, Anaflex 750, Disalcid, Marthritic, Mono-Gesic, Salflex, Salsitab); Sodium salicylate (various generics); Sulindac (Clinoril); Tolmetin sodium (Tolectin); and Valdecoxib (Bextra).

Preferably, the anti-inflammatory and/or analgesic agent agent comprises one or more selected from the following: Rapamycin, Tacrolimus, Ibuprofen, Ciclosporin, Diclofenac, Naproxen and related derivatives and salts thereof.

Most preferred the anti-inflammatory and/or analgesic agent Diclofenac and related derivatives and salts thereof. The Diclofenac may be in the form of Diclofenac potassium (Cataflam), Diclofenac sodium (Voltaren, Voltaren XR), or a Diclofenac salt in combination with another pharmaceutically active ingredient such as misoprostol (marketed under the Arthrotec brand).

If the anti-inflammatory and/or analgesic agent comprises Diclofenac and related derivatives and salts thereof, the average mean diameter may be in the approximate range of 50 to 250 nm. Preferably, the nanoparticles will have an average mean diameter in the range of 100 to 200 nm, more preferably the nanoparticles will have an average mean diameter in the range of 125 to 175 nm and most preferably an average mean diameter of about 150 nm and/or an average modal diameter of about 138 nm.

The nanoparticles may be formed with and/or in the presence of the anti-inflammatory agent. Various methods may be used to form the nanoparticles and it is envisaged that the nanoparticles will be formed as a polymer and anti-inflammatory and/or analgesic agent complex. However, polymer nanoparticles may be independently formed and then incubated with anti-inflammatory and/or analgesic agent so that it is absorbed or attached to the nanoparticles. Alternatively, the nanoparticles may be formed during incubation with anti-inflammatory and/or analgesic agent.

It will be apparent to the skilled addressee that the composition may further comprises one or more of the following component: buffers, excipients, binders, oils, water, emulsifiers, glycerin, antioxidants, preservatives and fragrances or any additional components usually found in topical creams, ointments sprays, powders, foams or mousses. Furthermore, the composition could be in a number of forms such as a paste or a suspension for use with a spraying device. Preferably, the composition is in the form of a topical medicament.

The dermatological condition will preferably comprise acne and/or atopic dermatitis or related conditions.

In accordance with another aspect of the present invention, there is provided use of polyhexamethylene biguanide (PHMB) to form one or more nanoparticles with, or associated with, an anti-inflammatory and/or analgesic agent in the preparation of a medicament for the treatment of a dermatological condition.

The nanoparticles may be used as the delivery vehicle for the anti-inflammatory and/or analgesic agents to an affected dermatological area.

The dermatological condition will preferably comprise acne and/or atopic dermatitis or related conditions.

In accordance with a further aspect of the present invention, there is provided a method of producing a composition for the treatment of a dermatological condition comprising mixing a polymer capable of forming nanoparticles with an anti-inflammatory and/or analgesic agent under conditions suitable to allow the formation of nanoparticles.

It is preferred that the method is used to produce a composition as herein above described.

In accordance with a further aspect of the present invention, there is provided a composition for use in the treatment of dermatological condition, comprising nanoparticles or nanoparticle conjugates formed of PHMB and an anti-inflammatory and/or analgesic agent.

In a related first aspect of the present invention, there is provided the use of nanoparticles or nanoparticle conjugates formed of PHMB and an anti-inflammatory and/or analgesic agent, for the manufacture or preparation of a medicament for the treatment of a dermatological condition.

PHMB (polyhexamethylene biguanide) is known as a safe and effective biocidal agent and is used as a sanitiser and preservative: U.S. Pat. Nos. 7,897,553, 4,758,595, US2008261841; US 20040009144. PHMB and related molecules are also found to be useful entry-promoting agents. It was surprisingly observed that PHMB (for example) itself enters a wide range of cells, including bacteria, fungi and mammalian cells. More surprisingly, PHMB (for example) is able to form nanoparticles with a wide range of molecules and deliver these molecules into such cells PCT/GB2012/052526. Finally the delivered molecules ranging from nucleic acids to small molecules were found to be functional inside cells. Moreover, work carried out with some natural product molecules such as retinoic acid and vitamin C have demonstrated an enhanced stabilizing effect on the natural products so they are less likely to break down when combined with PHMB.

Here we generally describe the invention of a formulation of anti-inflammatory and/or analgesic agents with PHMB which forms nanoparticles enabling penetration into and through the stratum corneum.

### DETAILED DESCRIPTION OF THE INVENTION

Embodiments of the present invention will now be described, by way of example only, with reference to the following experiments and accompanying figures, in which:

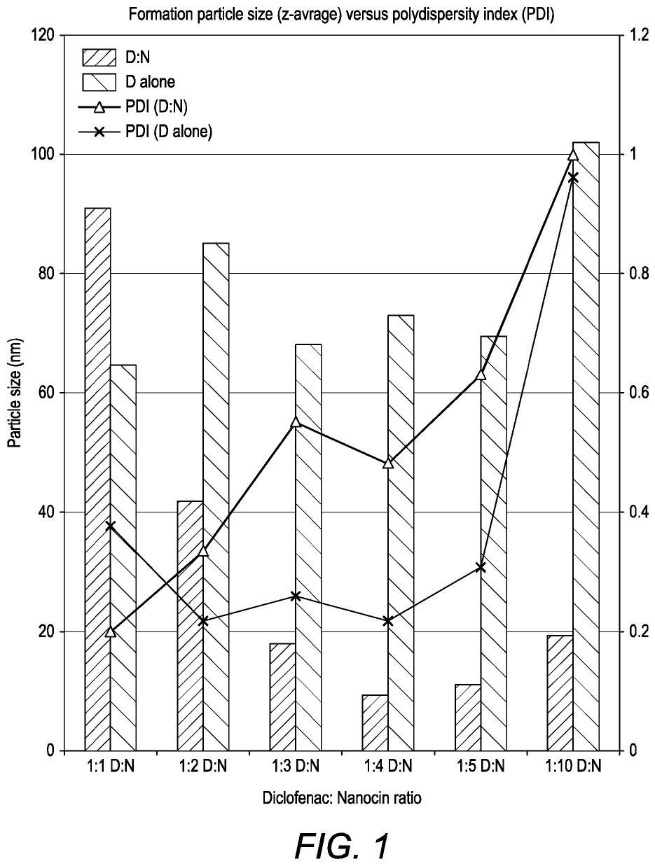

FIG. 1 is a graph, showing the formulation particle size (z-average) versus polydispersity index (PDI) of diclofenac and Nanocin as described in Example 1;

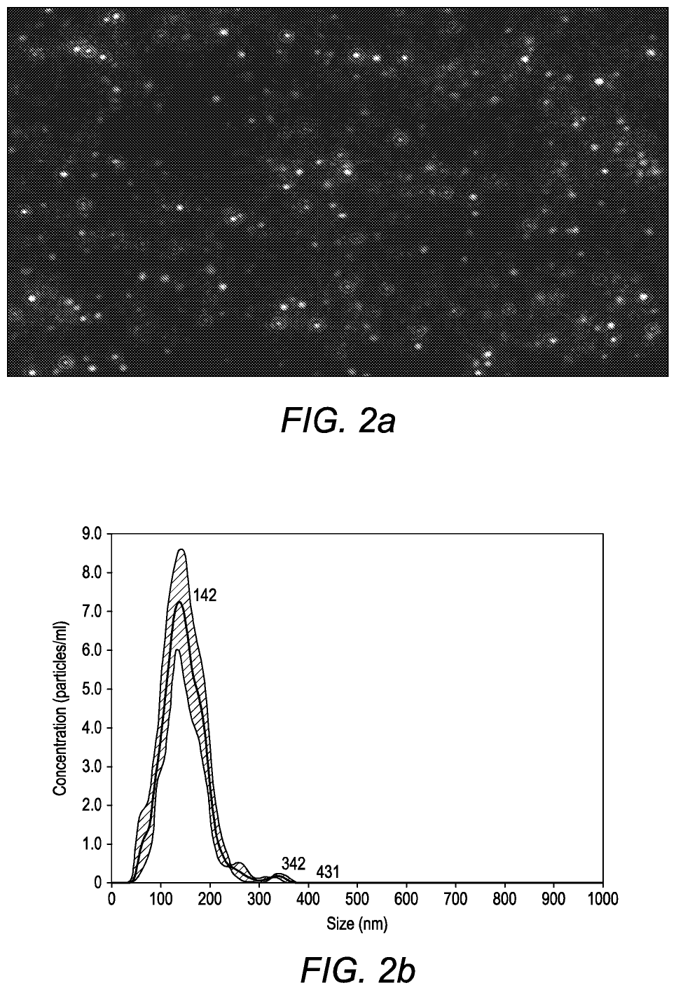

FIG. 2a is an image of LM10 capture Diclofenac and PHMB particles, whereas FIG. 2b is a graph showing the LM10 profile of the particle population versus size with the Diclofenac/Nanocin formulation in 20% ethanol as described in Example 1;

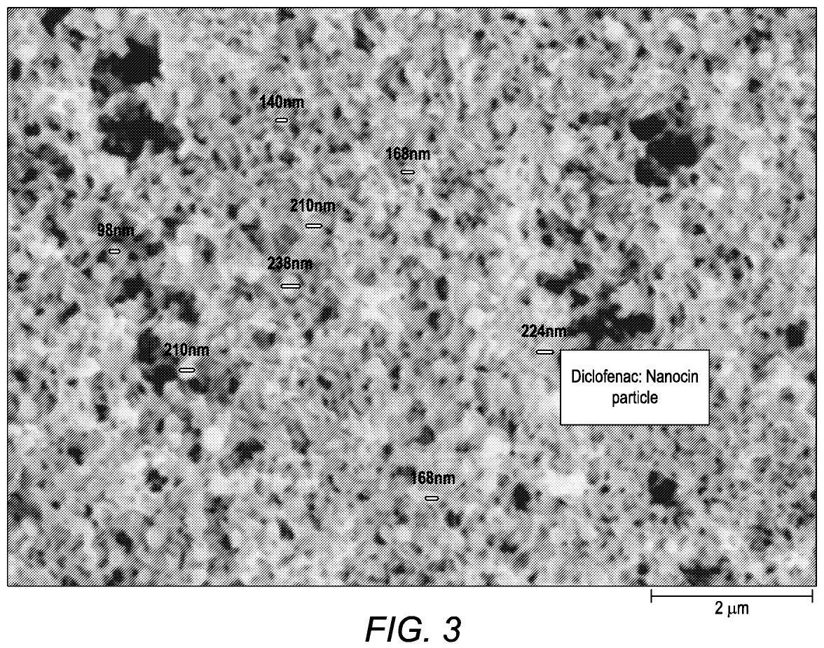

FIG. 3 shows a SEM micrograph of dehydrated diclofenac nanoparticles (imaged at 10 kV, 10 Kx Mag) as described in Example 1;

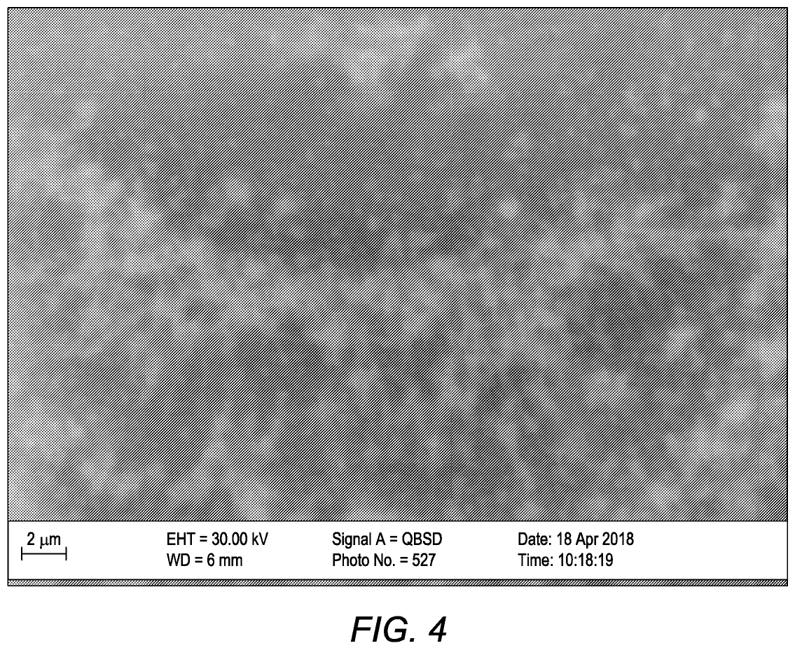

FIG. 4 shows a Backscatter image of nanoparticles in the WETSEM capsule (imaged at 30 kV, 4.6 kx Mag.) as described in Example 1;

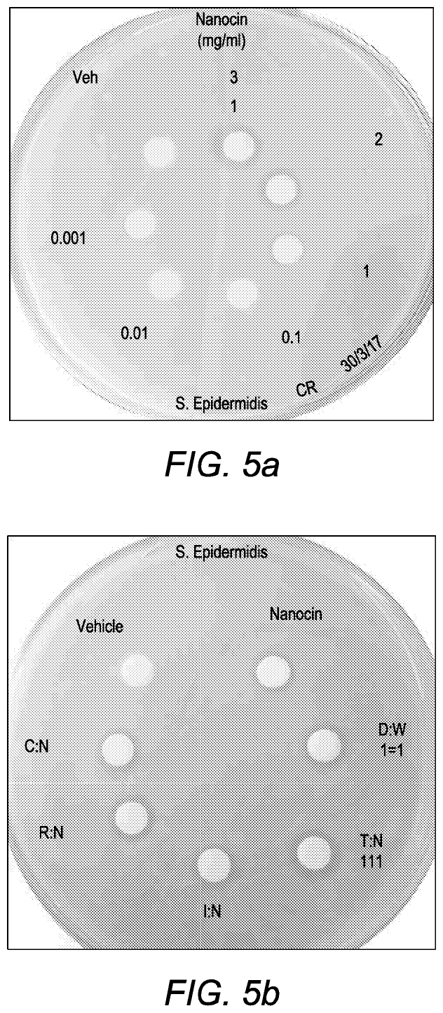

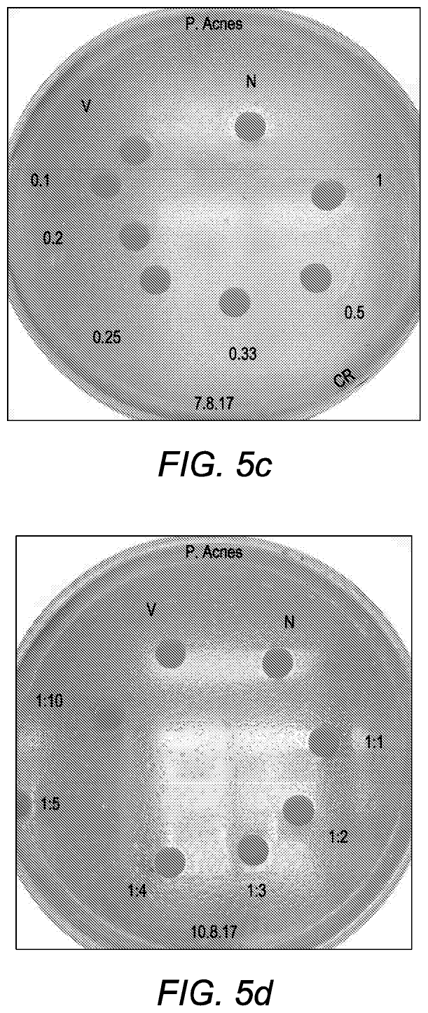

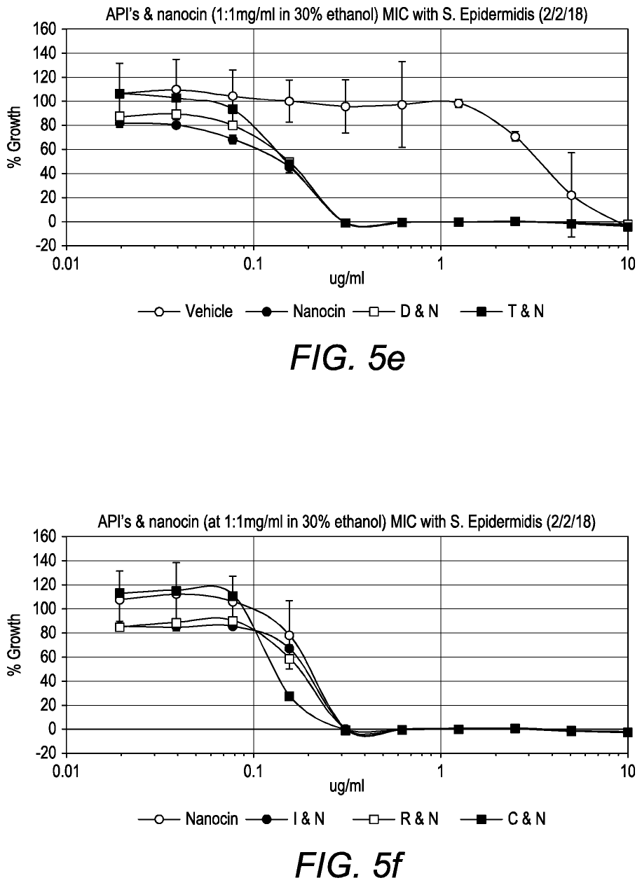

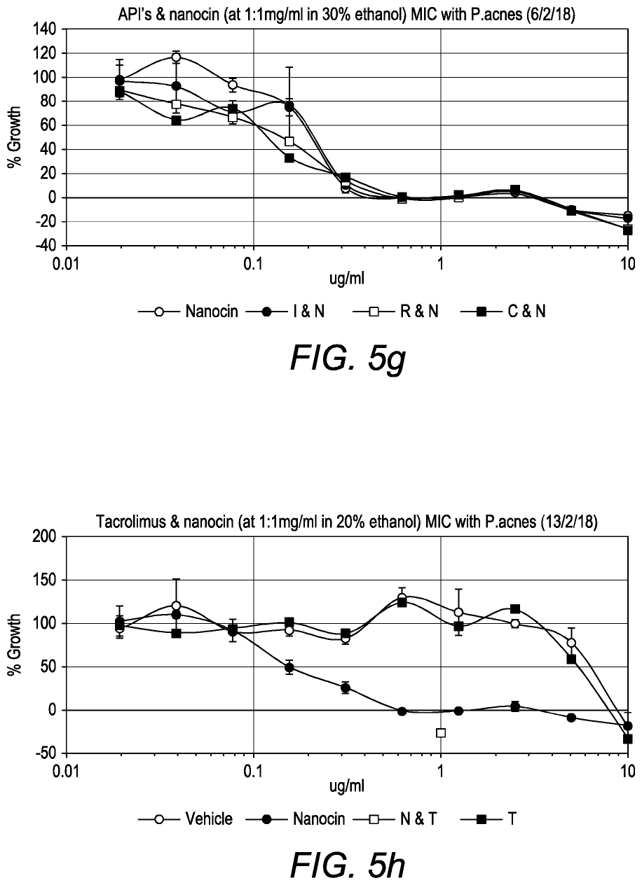

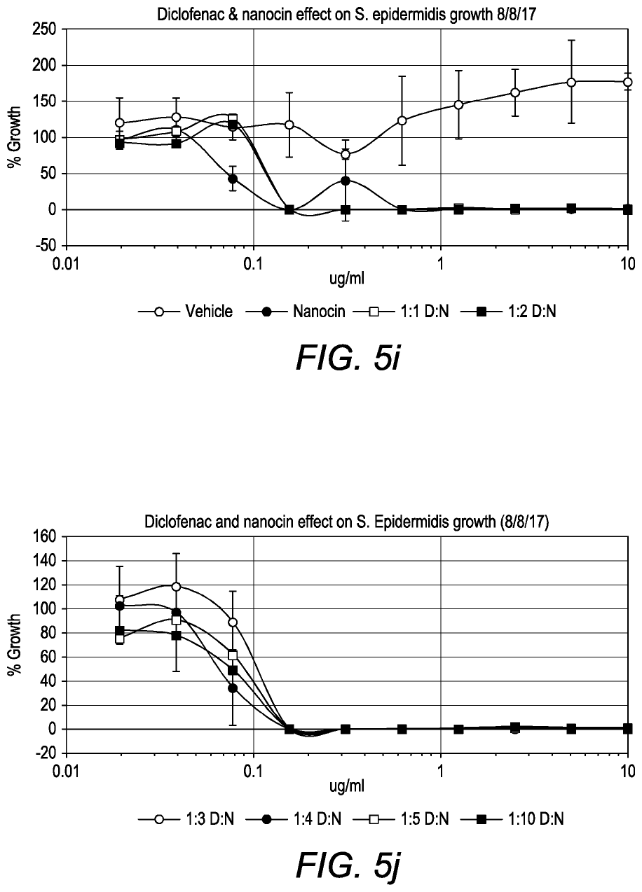

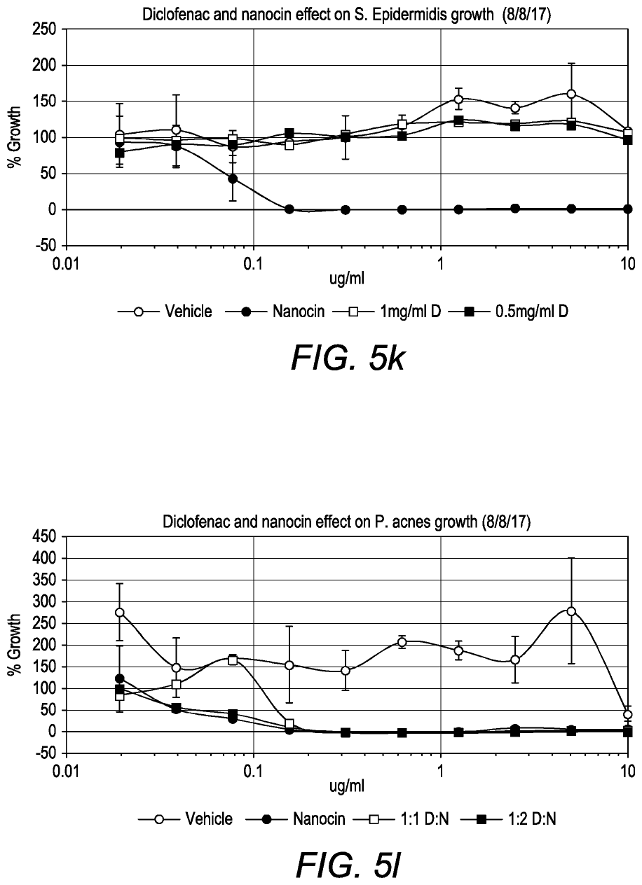

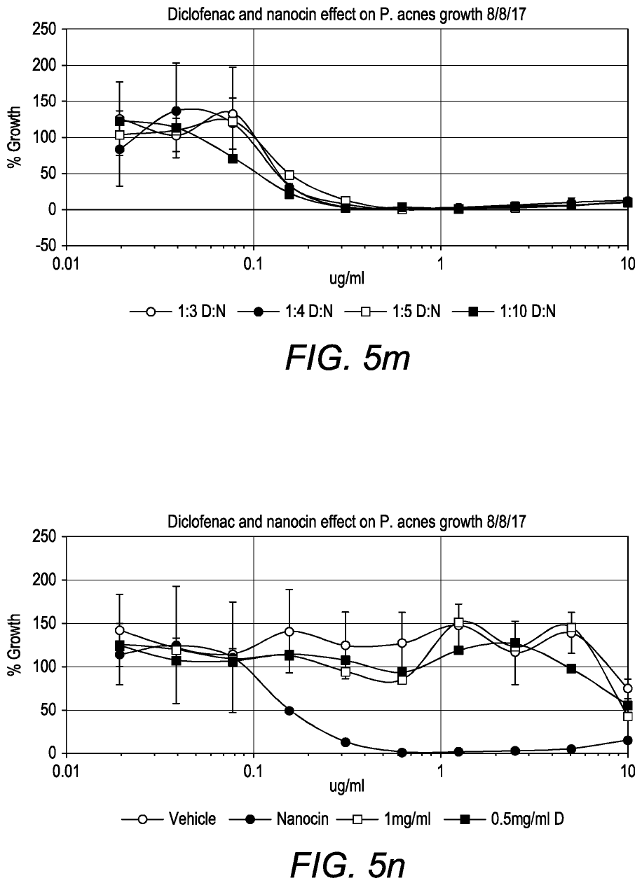

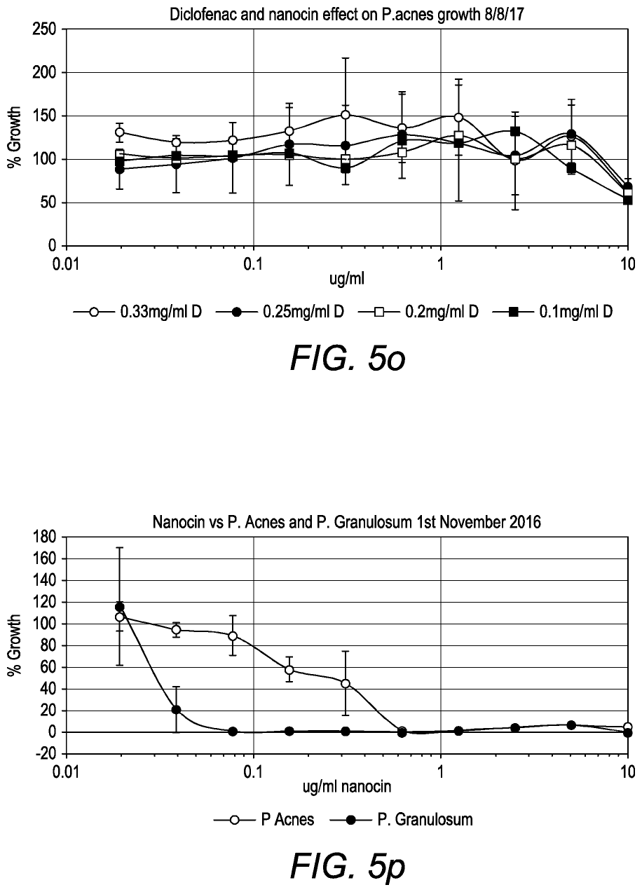

FIGS. 5a-d shows photographs of lawn assays as described in Example 1, FIG. 5a shows S. epidermidis lawn assay with Nanocin-soaked filter discs added to the cultures. Nanocin concentrations are in mg/ml. Vehicle was 20% ethanol, FIG. 5b. shows S. epidermidis lawn assay with either Nanocin (1 mg/ml) alone or Nanocin-API (1:1 mg/ml)—soaked filter discs, grown over 24 hours at 37° C. Vehicle was 20% ethanol, FIG. 5c. shows P. acnes lawn with Nanocin or Diclofenac-soaked filter discs (Diclofenac at 1, 0.5, 0.33, 0.25, 0.2 & 0.1 mg/ml. N-nanocin at 1 mg/ml, FIG. 5d. P. acnes lawn with Nanocin and Nanocin-Diclofenac formulations (Nanocin at a constant 1 mg/ml and Diclofenac reducing in concentration), FIGS. 5e-5i shows graphs detailing the growth of P. acnes and S. epidermidis when in the presence of various formulations as described in Example 1, FIG. 5e is a graph showing API's and nanocin (1:1 mg/ml in 30% ethanol) MIC with S. epidermidis, FIG. 5f is a graph showing API's and nanocin (1:1 mg/ml in 30% ethanol) MIC with S. epidermidis, FIG. 5g is a graph showing API's and nanocin (1:1 mg/ml in 30% ethanol) MIC with P. acnes, FIG. 5h is a graph showing Tacrolimus and nanocin (1:1 mg/ml in 20% ethanol) MIC with P. acnes, FIG. 5g is a graph showing API's & nanocin (at 1:1 mg/ml in 30% ethanol) MIC with P. acnes, FIG. 5h is a graph showing Tacrolimus & nanocin (at 1:1 mg/ml in 20% ethanol) MIC with P. acnes, FIG. 5i is a graph showing Diclofenac and nanocin effect on S. epidermidis growth, FIG. 5k is a graph showing Diclofenac and nanocin effect on S. epidermidis growth, FIG. 5j is a graph showing Diclofenac and nanocin effect on S. epidermidis growth, FIG. 5k is a graph showing Diclofenac and nanocin effect on S. epidermidis growth, FIG. 5l is a graph showing Diclofenac and nanocin effect on P. acnes growth, FIG. 5m is a graph showing Diclofenac and Nanocin effect on P. acnes growth, FIG. 5n is a graph showing Diclofenac and Nanocin effect on P. acnes growth, FIG. 5o is a graph showing Diclofenac and nanocin effect on P. acnes growth, and FIG. 5p is a graph showing Nanocin vs P. Acnes and P. Granulosum;

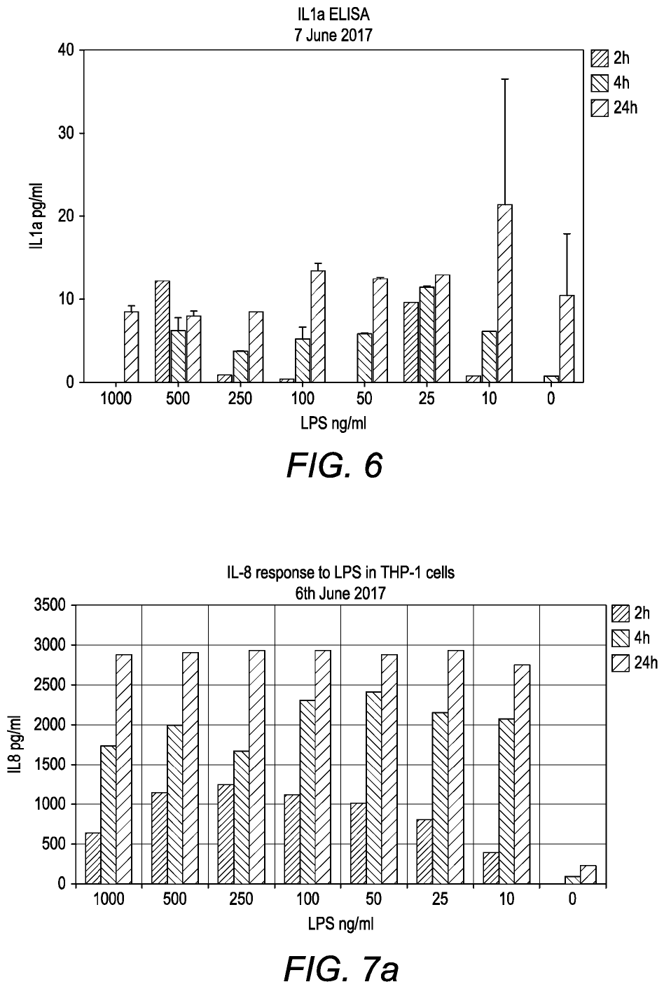

FIG. 6 is a graph showing the LPS dose (0-1 ug/ml) investigated on TNF-α, IL-8 and IL-1α response, over a time period from 2,4 and 24 hours as described in Example 1;

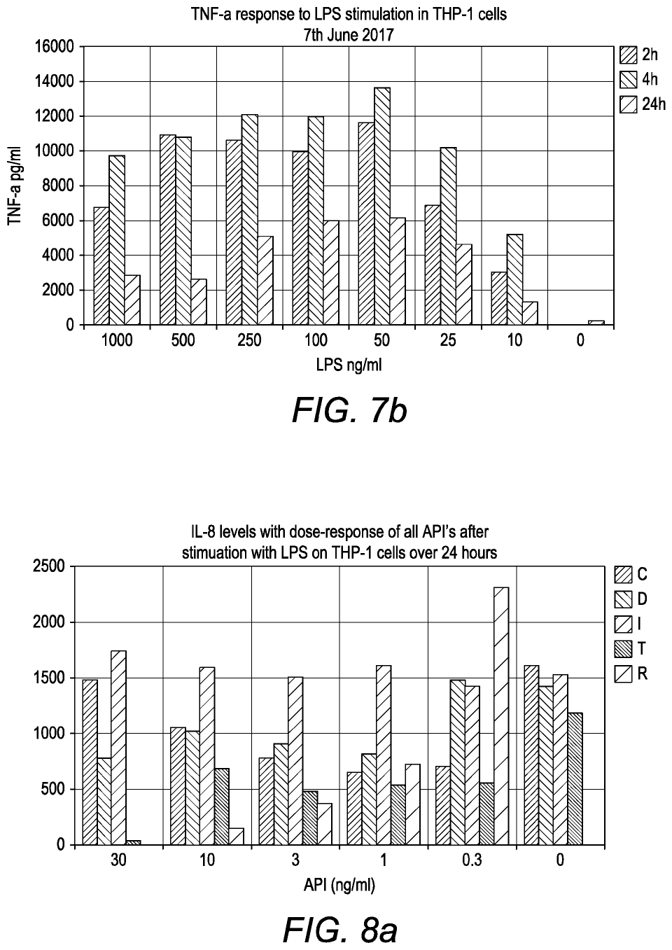

FIG. 7a is a graph showing IL-8 response to LPS in THP-1 cells over 2 h, 4 h and 24 h, FIG. 7b is a graph showing TNF-α response to LPS stimulation in THP-1 cells over 2 h, 4 h and 24 h as described in Example 1;

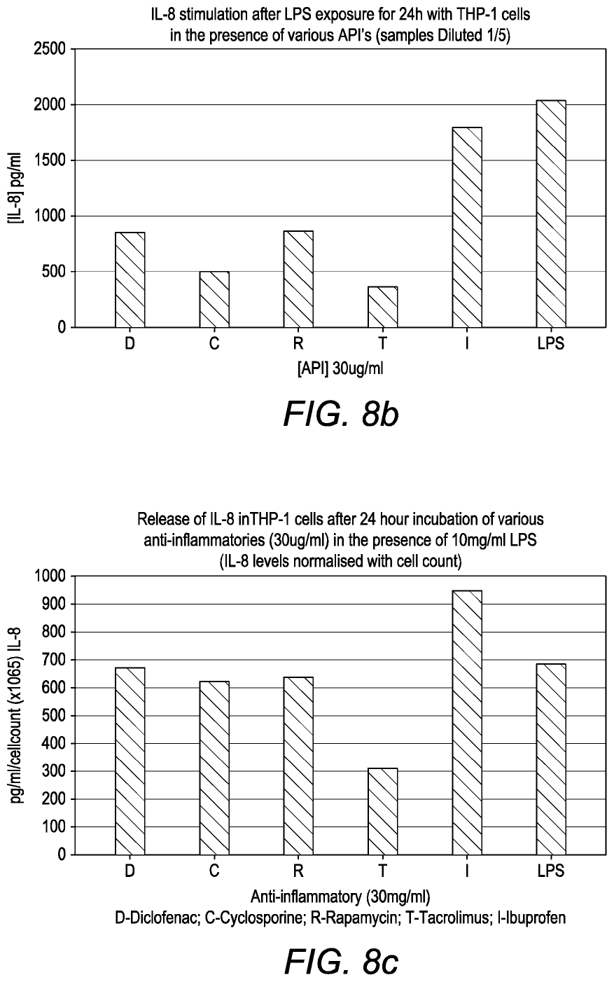

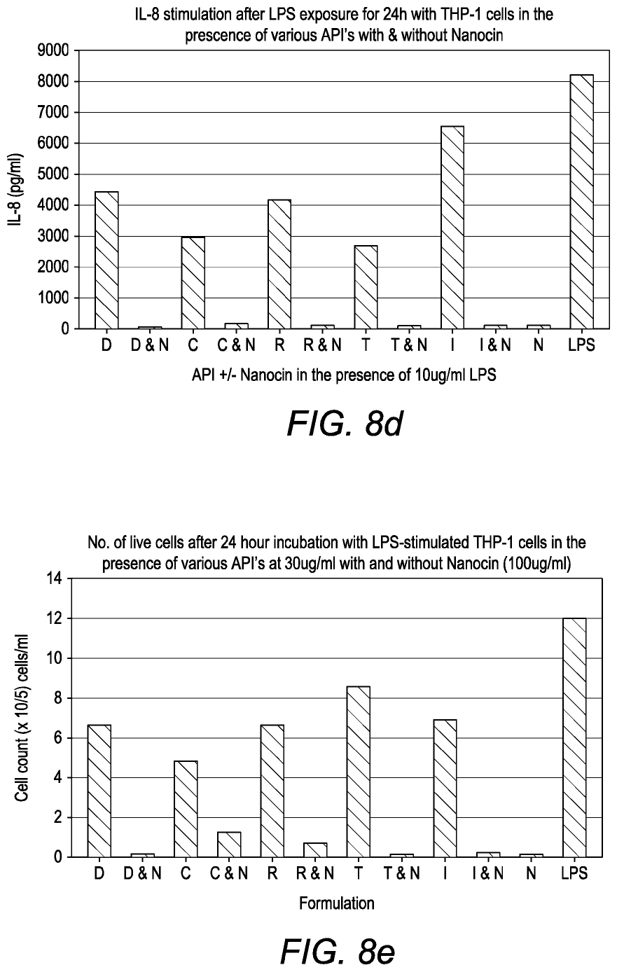

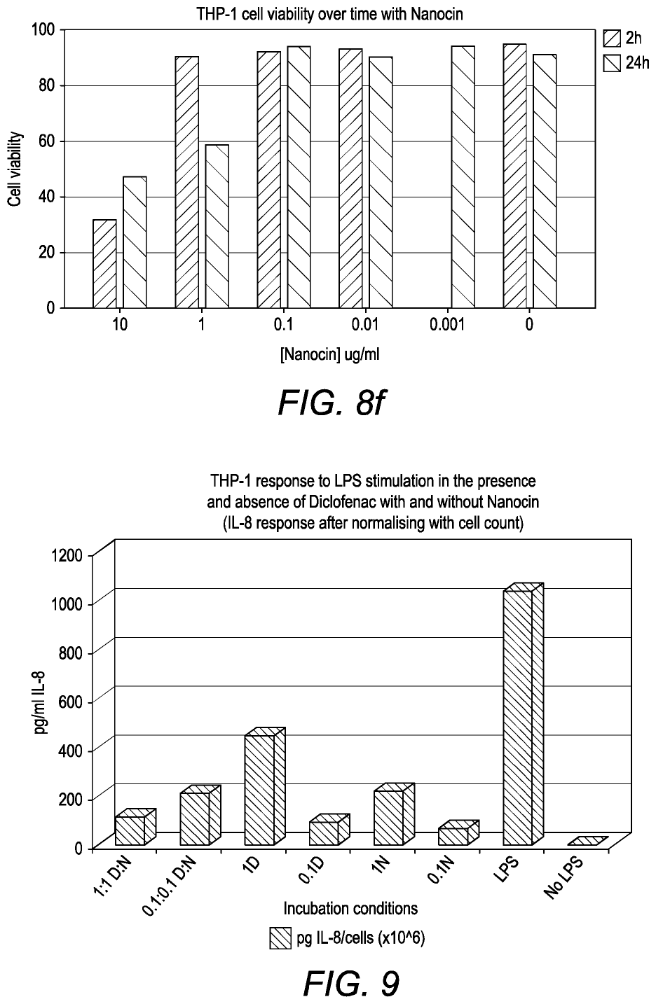

FIG. 8a is a graph showing IL-8 levels with dose-response of all APIs after stimulation with LPS on THP-1 cells over 24 h, FIG. 8b is a graph showing IL-8 stimulation after LPS exposure for 24 h with THP-1 cells in the presence of APIs (where the samples are diluted ⅕), FIG. 8c is a graph showing the release of IL-8 in THP-1 cells after a 24 hour incubation of various anti-inflammatories (30 ug/ml) in the presence of 10 ug/ml LPS (IL-8 levels normalised with cell count), FIG. 8d is a graph showing IL-8 stimulation after LPS exposure for 24 hours with THP-1 cells in the presence of various APIs with and without Nanocin, FIG. 8e is a graph showing number of live cells after 24 hour incubation with LPS-stimulated THP-1 cells in the presence of various APIs at 30 ug/ml with and without Nanocin (100 ug/ml), FIG. 8f is a graph showing THP-1 cell viability over time with Nanocin at 2 h and 24 h;

FIG. 9 is a graph showing THP-1 response to LPS stimulation in the presence and absence of Diclofenac with and without Nanocin (IL-8 response after normalising with cell count) as described in Example 1;

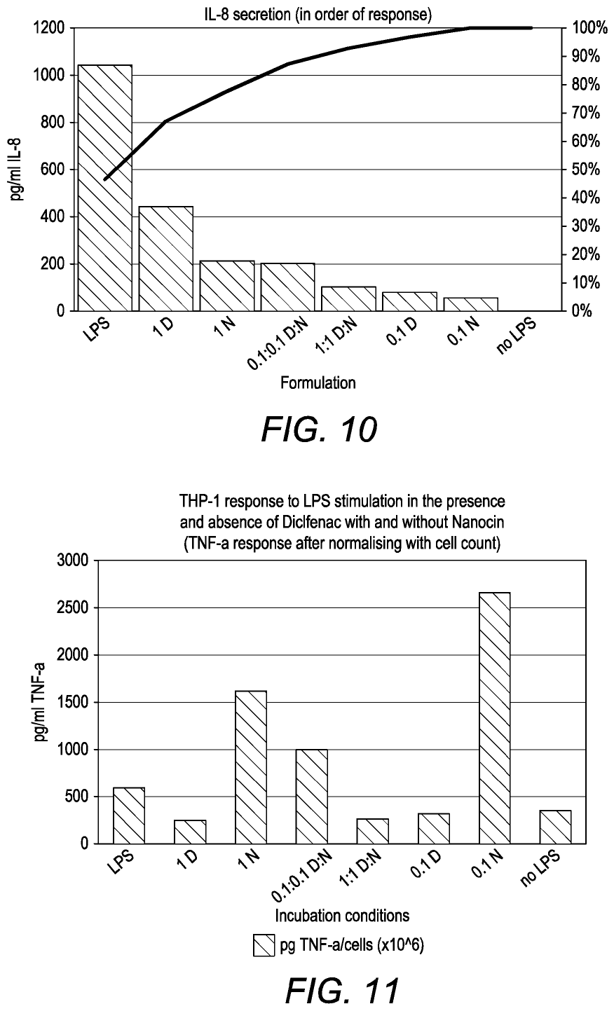

FIG. 10 is a graph showing IL-8 secretion (in order of response) as described in Example 1;

FIG. 11 is a graph showing THP-1 response to LPS stimulation in the presence and absence of Diclofenac with and without Nanocin (TNF-α response after normalising with cell count) as described in Example 1;

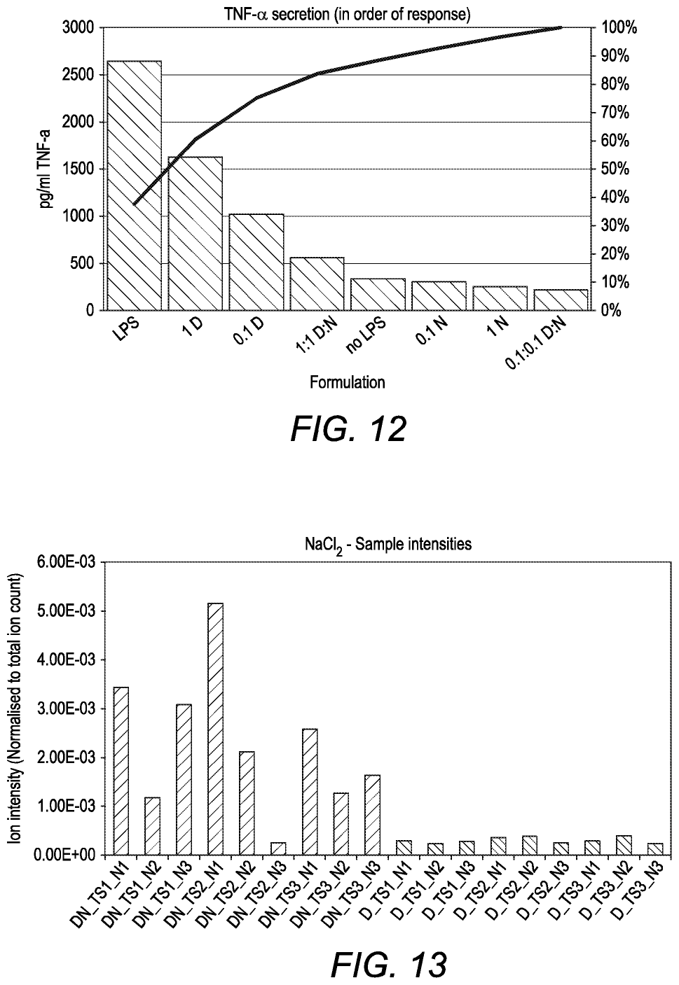

FIG. 12 is a graph showing TNF-α secretion (in order of response) as described in Example 1;

FIG. 13 is a graph showing NaCl₂ sample intensities as described in Example 1;

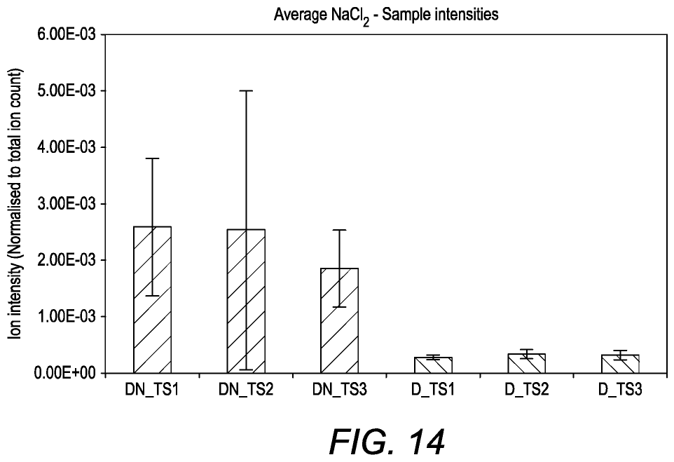

FIG. 14 is a graph showing average NaCl₂ sample intensities as described in Example 1;

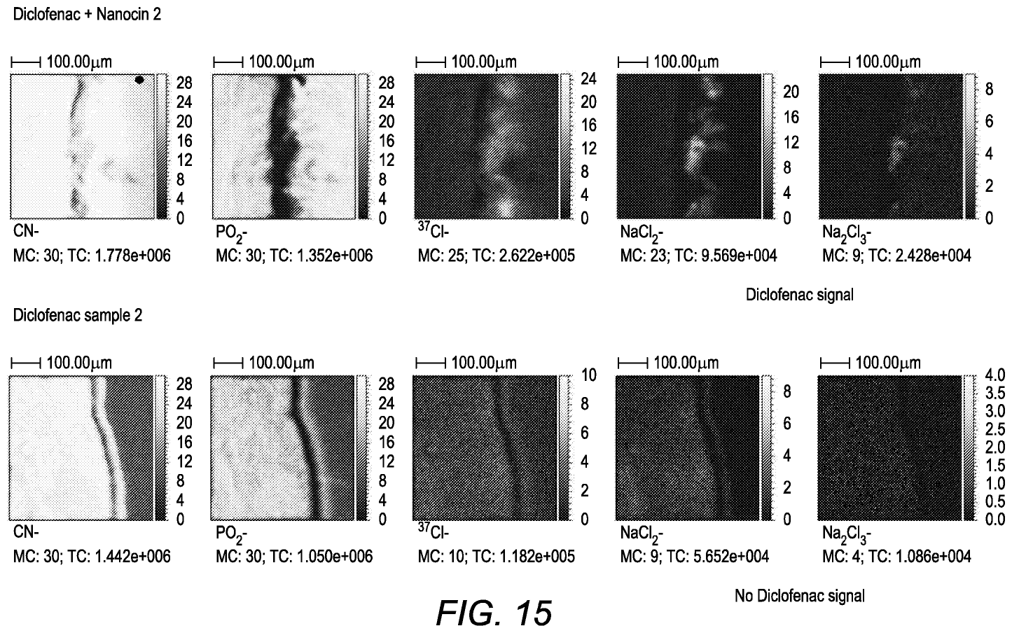

FIG. 15 are cross-sectional images showing permeation into the stratus corneum of diclofenac+Nanocin and diclofenac alone as described in Example 1;

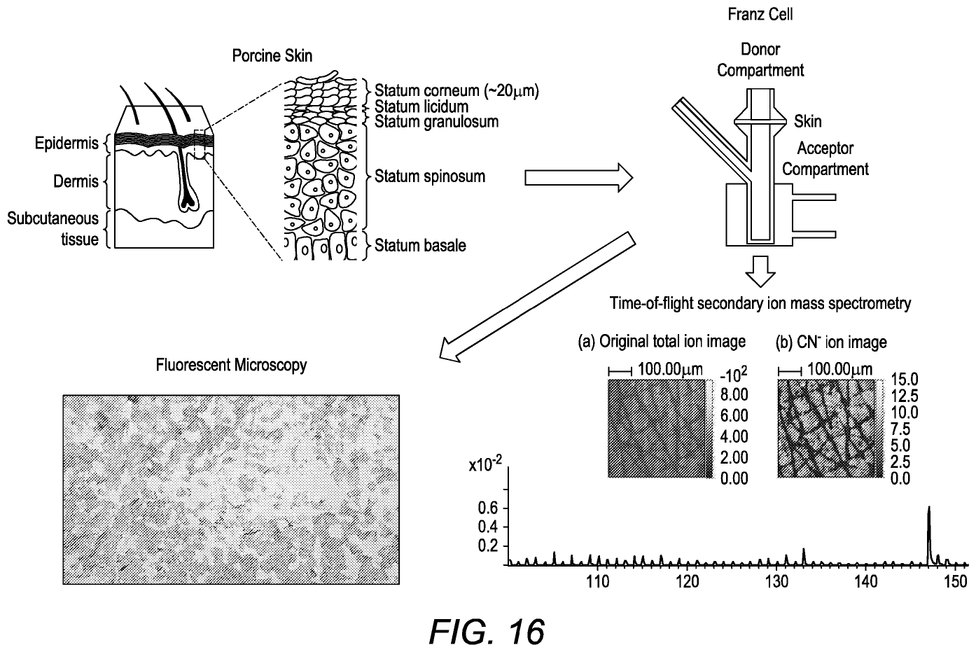

FIG. 16 shows a schematic diagram of the sample preparation for chemical imaging utilised in Example 2;

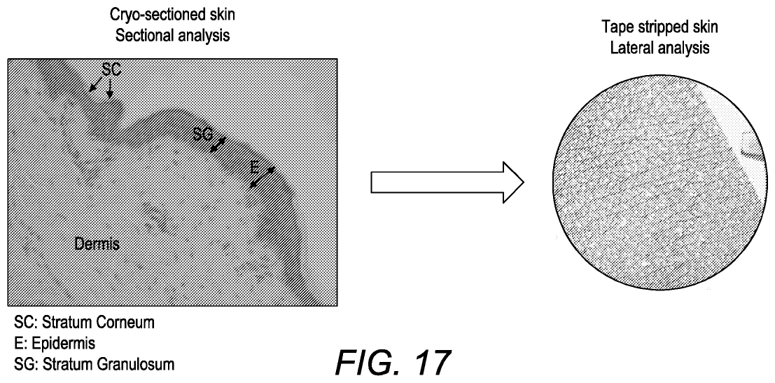

FIG. 17 shows a cross sectional analysis vs tape strip analysis utilised in Example 2;

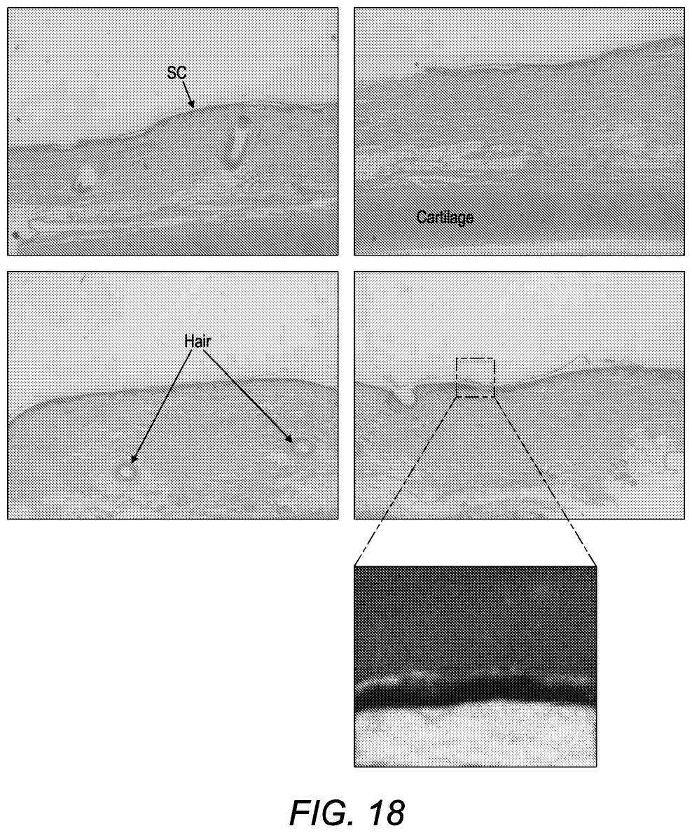

FIG. 18 shows Example Section Images (H&E Stained) described in Example 2;

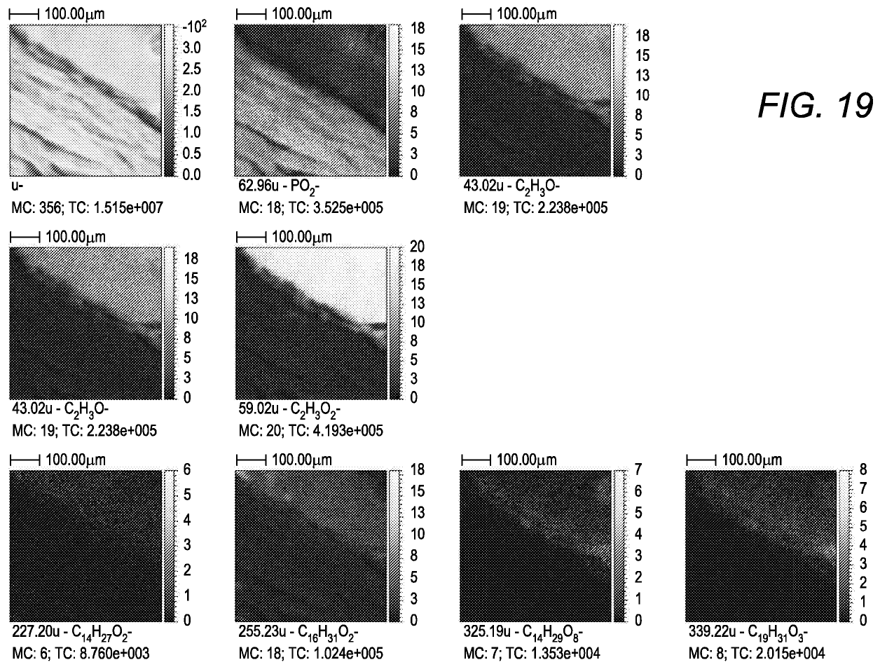

FIG. 19 shows cross sectional analysis using ToF-SIMS chemical imaging for API+Nanocin as described in Example 2;

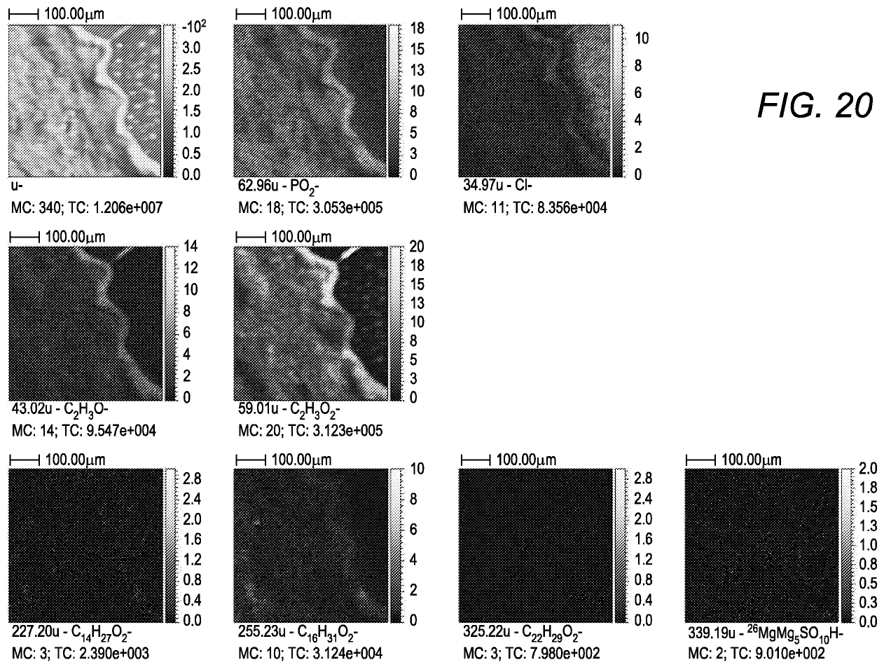

FIG. 20 shows cross sectional analysis using ToF-SIMS chemical imaging for Tacrolimus+Nanocin as described in Example 2;

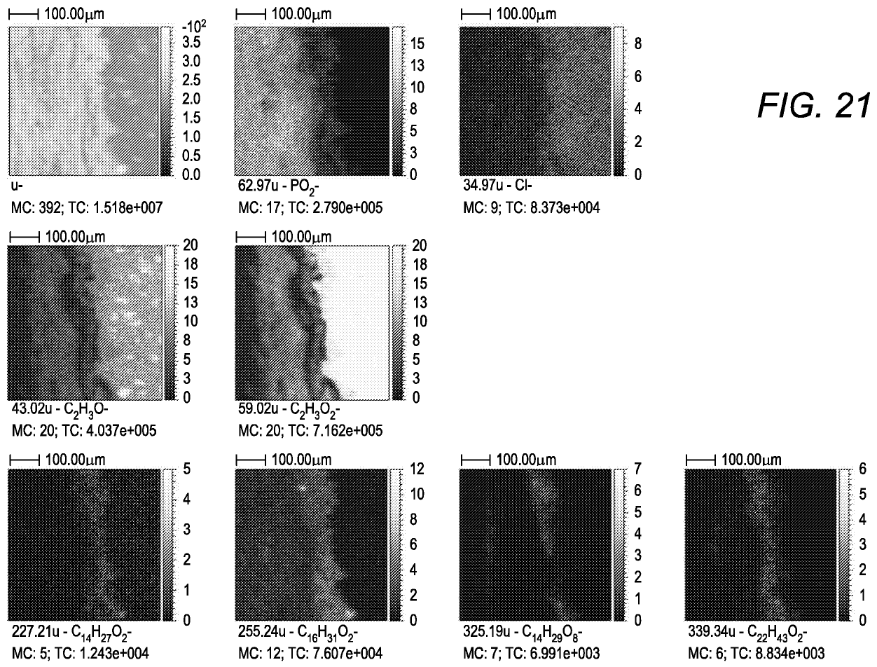

FIG. 21 shows cross sectional analysis using ToF-SIMS chemical imaging for Diclofenac+Nanocin as described in Example 2;

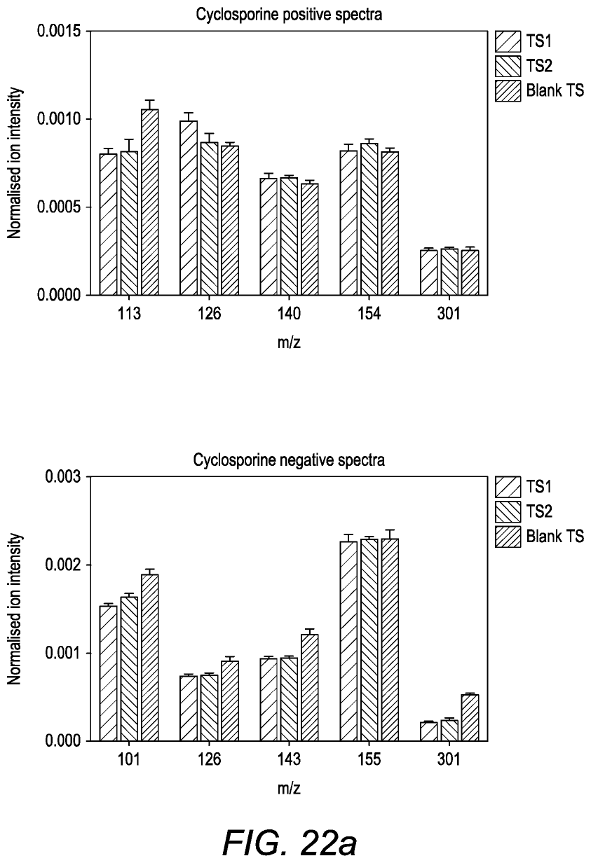

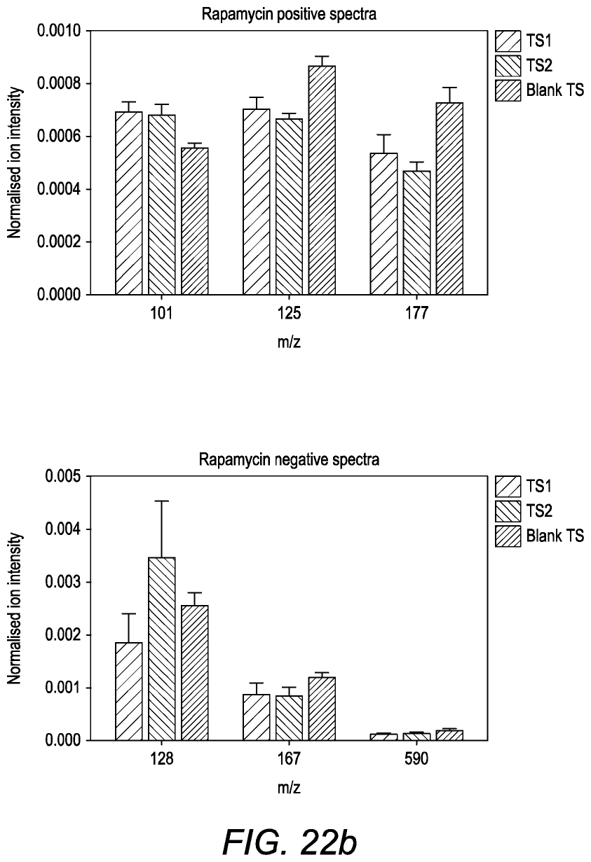

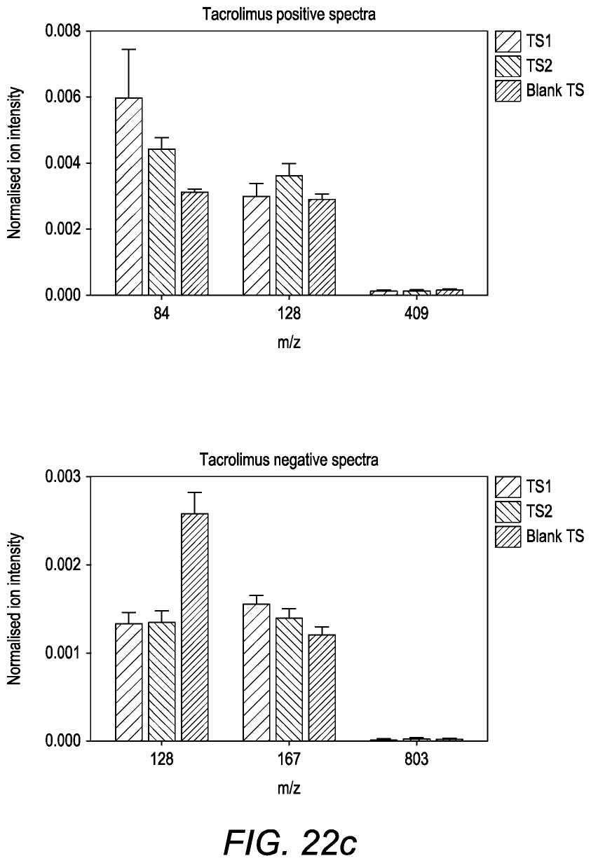

FIG. 22a-22c shows graphs showing API+tape strip analysis as described in Example 2, FIG. 22a shows Cyclosporine+Nanocin in positive and negative spectra, FIG. 22b shows Rapamycin+Nanocin in positive and negative spectra, and FIG. 22c shows Tacrolimus+Nanocin in positive and negative spectra;

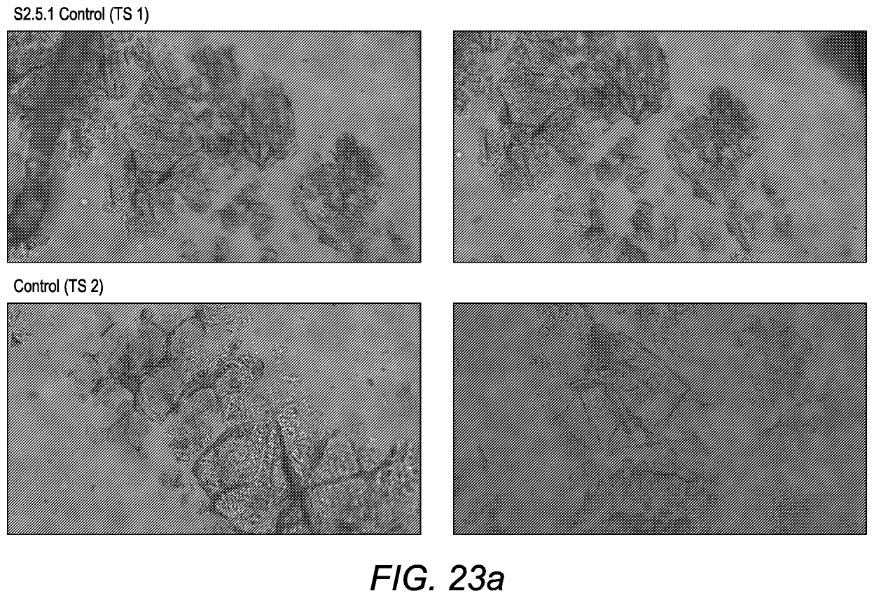

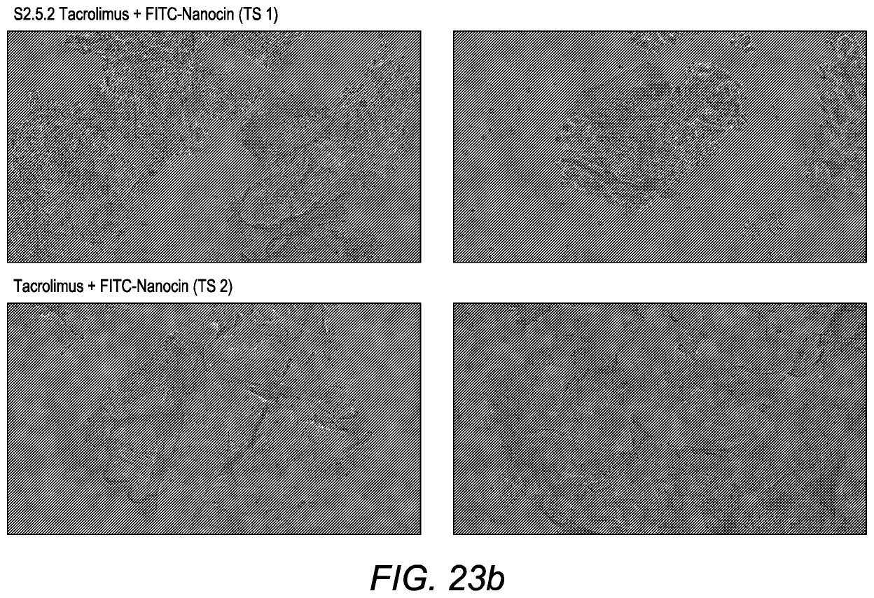

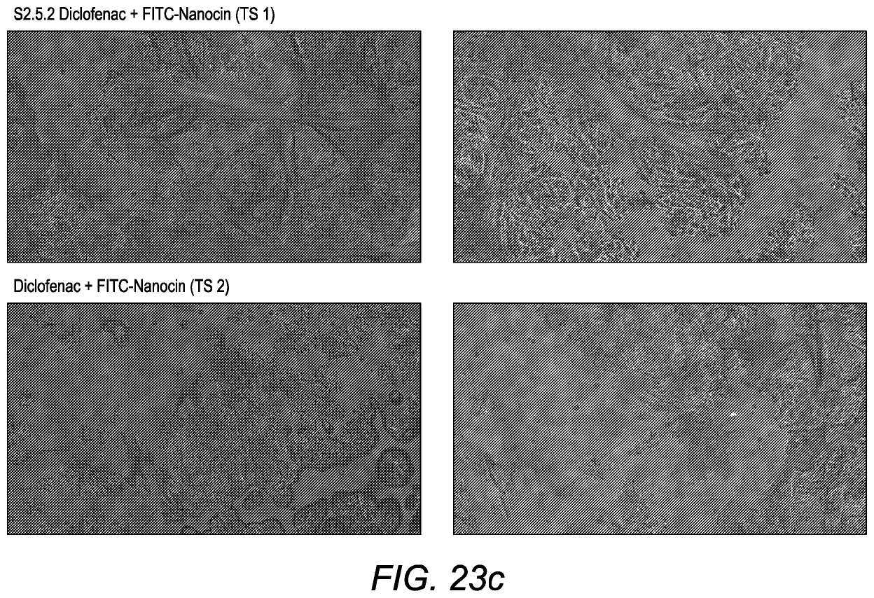

FIG. 23a-23c shows fluorescence micrographs of the API+FITC-Nanocin tape strip analysis described in Example 2, FIG. 23a shows micrographs for controls TS 1 and TS 2, FIG. 23b shows micrographs for Tacrolimus+FITC-Nanocin for TS 1 and TS 2,

FIG. 23c shows micrographs for Diclofenac+FITC-Nanocin for TS 1 and TS 2;

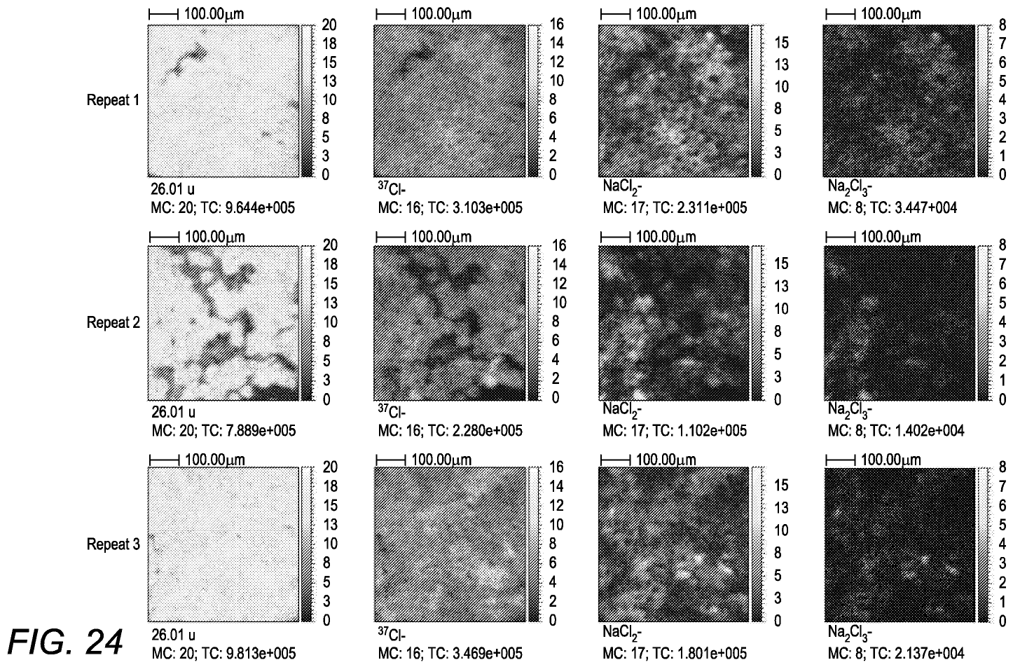

FIG. 24 shows cross sectional analysis using ToF-SIMS chemical imaging for Diclofenac+Nanocin TS 1 as three repeats as described in Example 2;

FIG. 24 shows cross sectional analysis using ToF-SIMS chemical imaging for Diclofenac+Nanocin TS 2 as three repeats as described in Example 2;

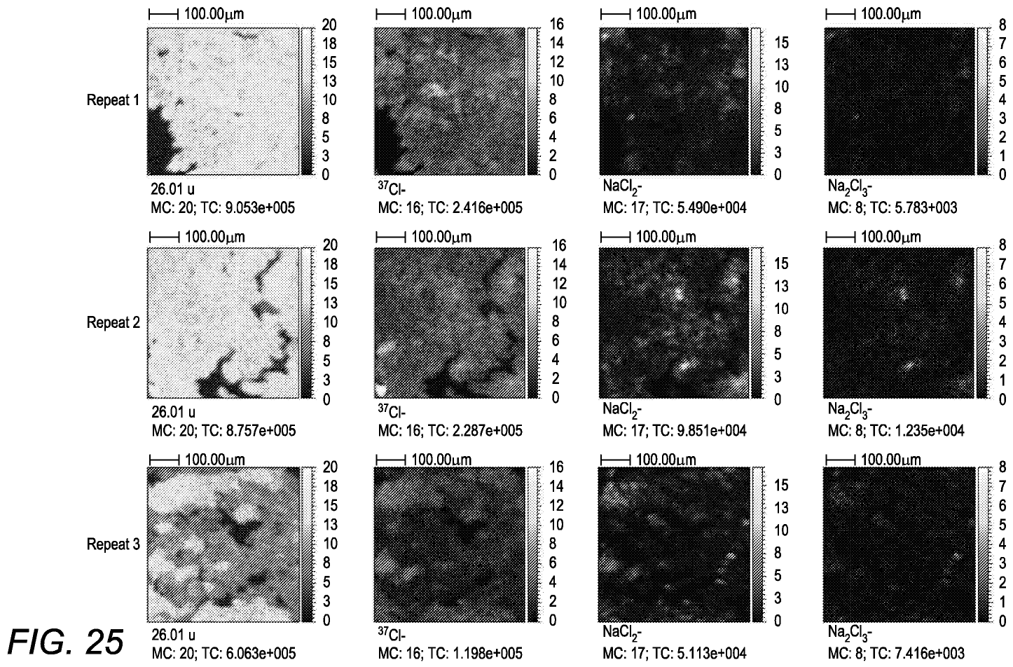

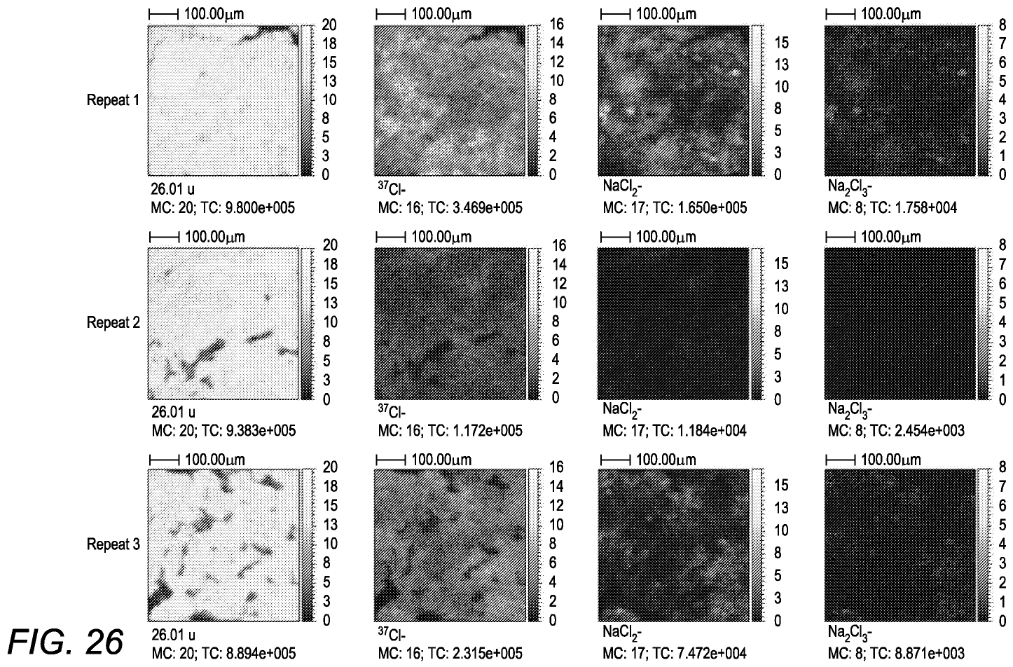

FIG. 25 shows cross sectional analysis using ToF-SIMS chemical imaging for Diclofenac+Nanocin TS 2 as three repeats as described in Example 2;

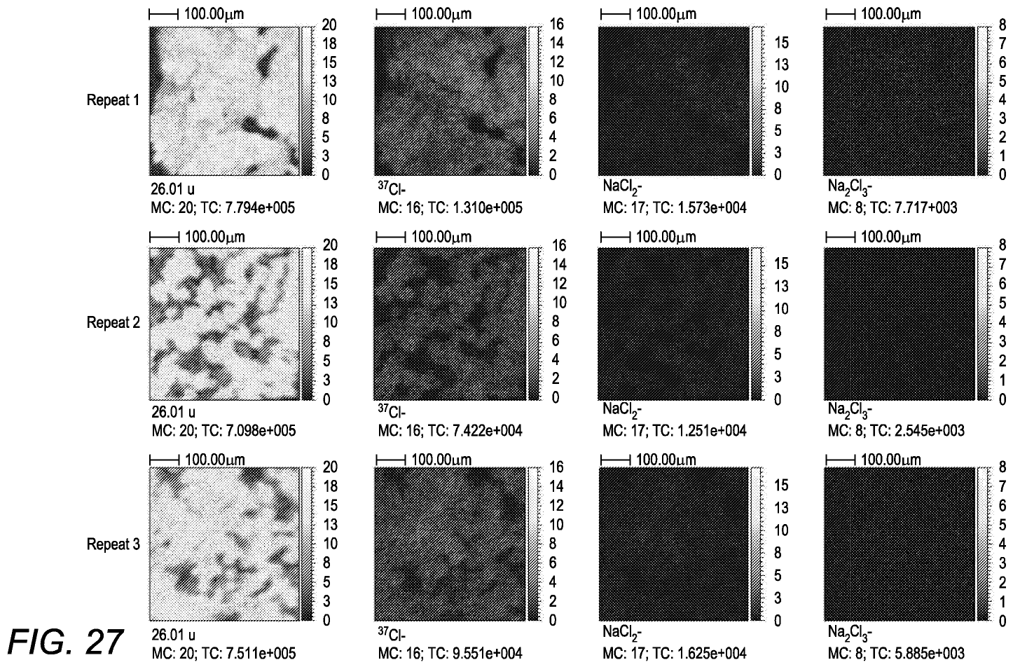

FIG. 26 shows cross sectional analysis using ToF-SIMS chemical imaging for Diclofenac+Nanocin TS 3 as three repeats as described in Example 2;

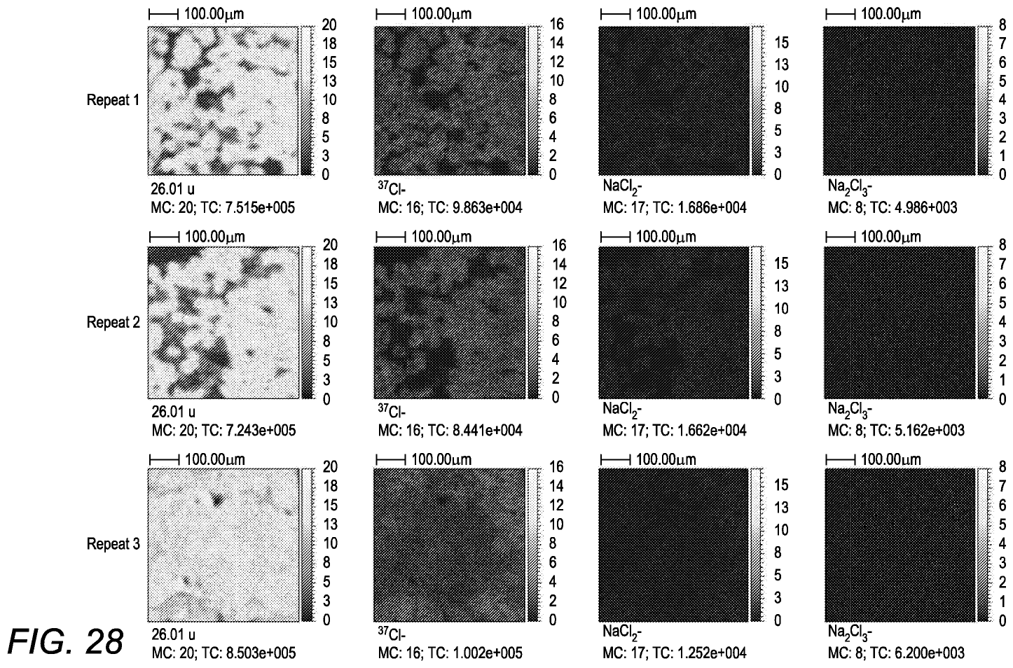

FIG. 27 shows cross sectional analysis using ToF-SIMS chemical imaging for Diclofenac TS 1 as three repeats as described in Example 2;

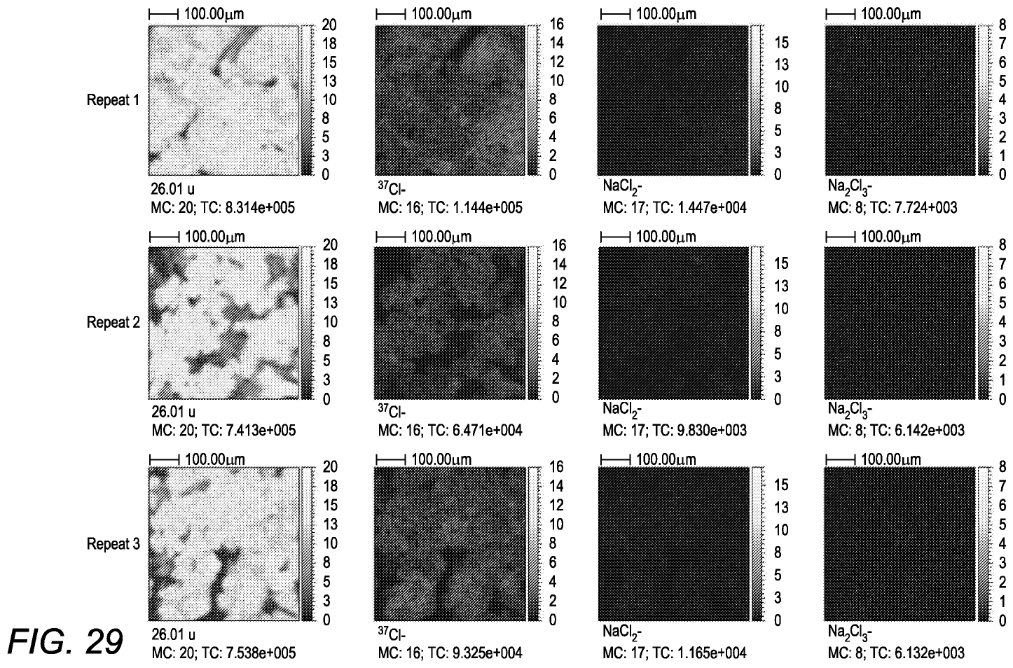

FIG. 28 shows cross sectional analysis using ToF-SIMS chemical imaging for Diclofenac TS 2 as three repeats as described in Example 2;

FIG. 29 shows cross sectional analysis using ToF-SIMS chemical imaging for Diclofenac TS 3 as three repeats as described in Example 2;

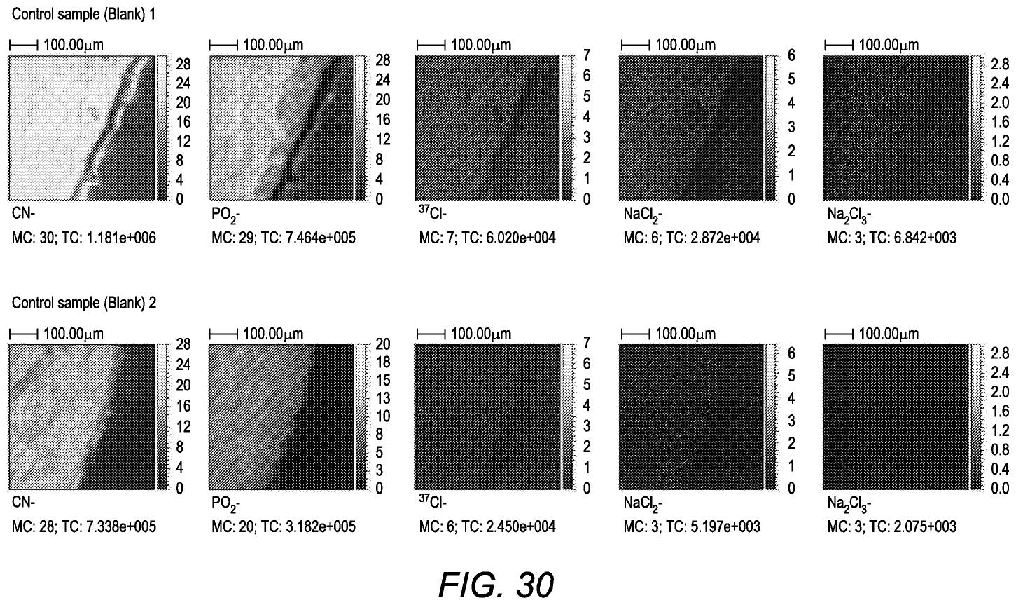

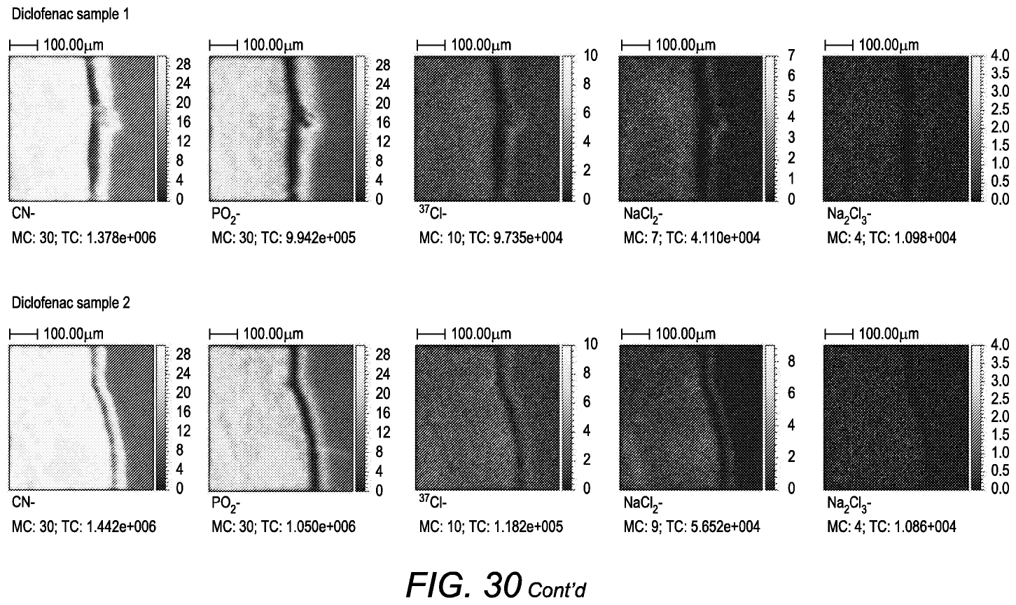

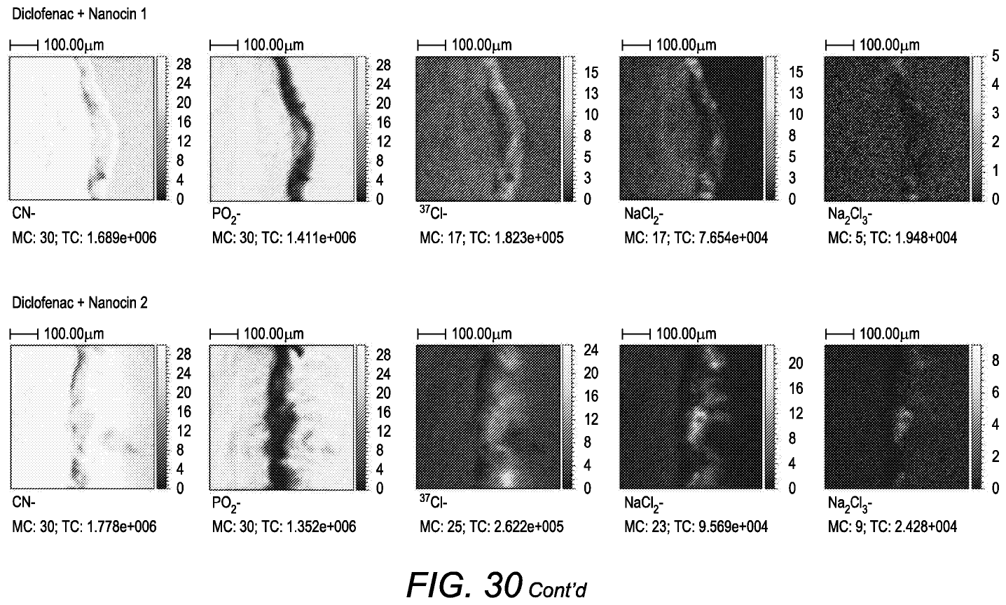

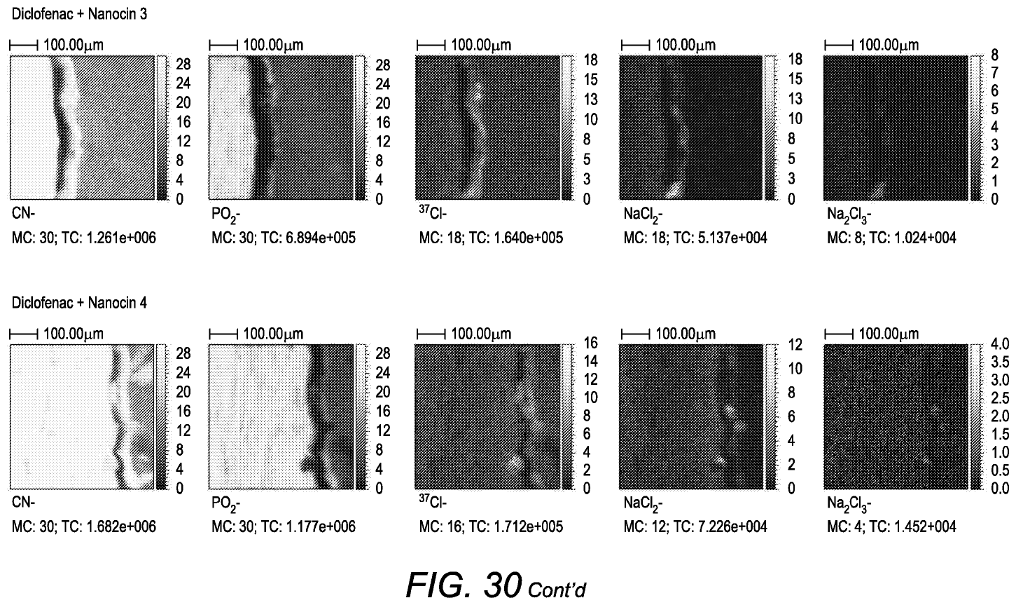

FIG. 30 shows cross sectional analysis using ToF-SIMS chemical imaging as described in Example 2 for a) Control Sample (Blank) 1, b) Control Sample (Blank) 2, c) Diclofenac Sample 1, d) Diclofenac Sample 2, e) Diclofenac+Nanocin 1, f) Diclofenac+Nanocin 2, g) Diclofenac+Nanocin 3, and h) Diclofenac+Nanocin 4;

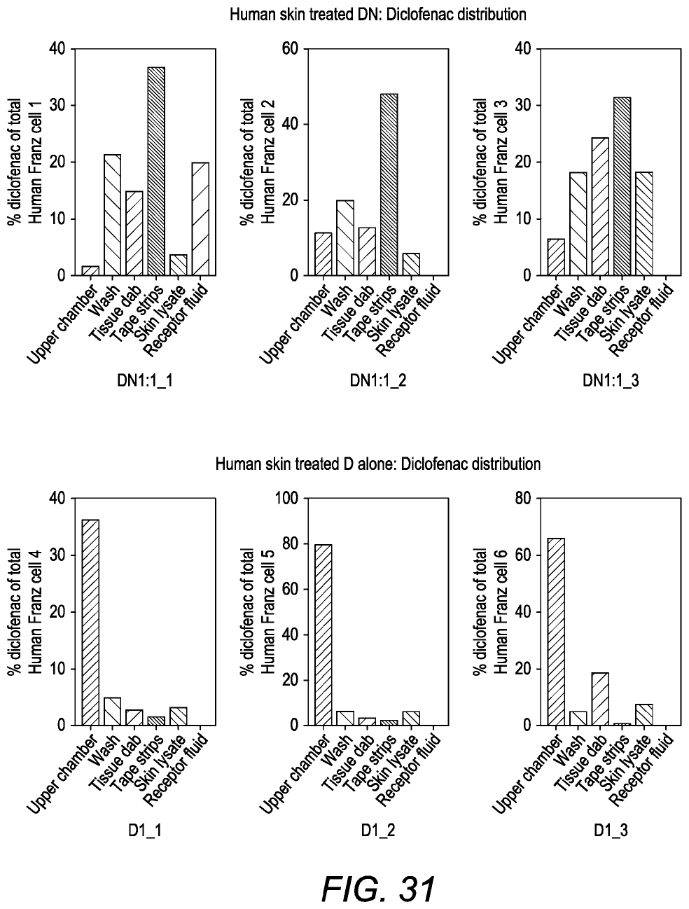

FIG. 31 show graphs of human vs pig diclofenac and nanocin distributions by %. Samples were analysed by quantitate LC-MS for the presence of diclofenac. The proportion of the drug found in each sample was calculated compared to the total amount that had been applied to the upper chamber of the Franz cell (%); and

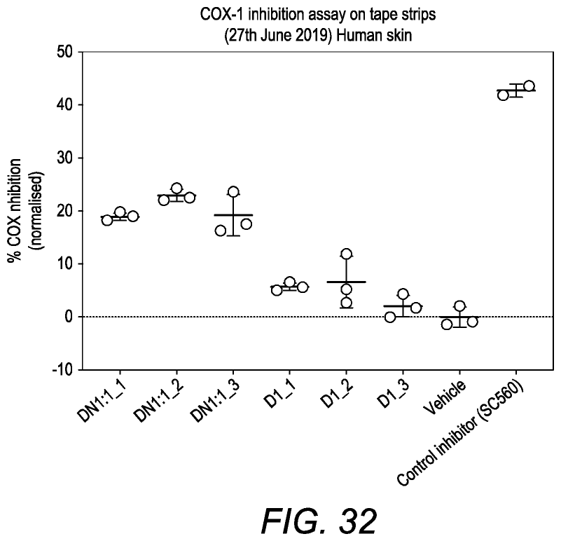

FIG. 32 is a graph showing the inhibition of cyclooxygenase 1 in the human skin studies. Cyclooxygenase-1 (Cox-1) inhibition was determined using an assay kit from Abcam according to the manufacturer's instructions. The % inhibition of Cox-1 was determined and normalised to the average Vehicle alone treatment.

### EXAMPLES

### Example 1—Drug Reformulation of Anti-Inflammatories with Nanocin as a Therapeutic Approach for Acne

### Background

A program of work was chosen to screen a number of anti-inflammatories formulated with Nanocin® (Tecrea Ltd, UK) (polyhexamethylene biguanide (PHMB)) to determine which would be best to take forward as an acne remedy. The active pharmaceutical ingredient (API)/Nanocin selection process was determined by the following program of work:
Solubility in water and ethanol vehicles.
Formulation of the chosen API's with Nanocin, looking at particle formation, size and quality.
Use electron microscopy to confirm nanoparticle formation of interesting formulations.
Measuring the anti-microbial activity of nanocin with acne-relevant bacteria and qualify that the API's do not antagonise this effect.
Measuring the anti-inflammatory activity of the API's and qualify that nanocin does not antagonise this effect.

Topical skin application studies were also used to determine if formulating the API's enhances delivery of the API's into the skin.

Five anti-inflammatories of different classifications were chosen for the initial screening in the acne program and are detailed in Table 1 below.

TABLE 1

| | | | Inhibitory |
|---|---|---|---|
| | | | Effect |
| Anti- | | | (relevant to |
| Inflammatory | Indication | Type | acne) |
| Cyclosporine A | Immunosuppressant- | Calcineurin | TNF-α, IL-8, IL-1α |
| | Organ rejection | Inhibitor | |
| Tacrolimus | Atopic dermatitis | Calcineurin | Down |
| (Protopic) | | Inhibitor | regulates IL-8 |
| | | | receptor in |
| | | | keratinocytes; |
| | | | TNF-α |
| Rapamycin | Immunosuppressant- | MTOR | IL-8 |
| (Sirolimus) | Organ rejection | inhibitor | |
| Diclofenac | Arthritis | COX 2 | IL-1α |
| | | inhibitor | |
| Celebrex | Arthritis | COX 2 | IL-1α |
| | | inhibitor | |

Solubilites of each compound in ethanol & water was determined and are shown in Table 2 below:

TABLE 2

| % | 10 mg/ml API solution | | | | Cele- |
|---|---|---|---|---|---|
| Ethanol | Cyclosporine | Tacrolimus | Rapamycin | Diclofenac | coxib |
| 5 | x | x | x | Y | x |
| 10 | x | x(better) | x | y | x |
| 15 | x | y | x | y | x |
| 20 | x | y | x | y | x |
| 30 | y | y | y | y | x |
| water | x | x | x | y | x |

(where x = not souble and y = soluble)

Celcoxib was then dropped from the program due to its insolubility, but Ibuprofen (a non-selective COX inhibitor, with better solubility in water and ethanol) was tested as a replacement.

### Formulation Work

As Diclofenac (D) was the most soluble, it was formulated first with Nanocin. A ratio of Diclofenac with Nanocin (D:N) was tested and showed change in particle size with the differing ratios. However, the polydispersity index (a measure of the variability of nanoparticle size in the mixture), as shown in FIG. 1, was reported as ‘good’ only in the 1:1 mg/ml mixture.

A 1:1 mg/ml ratio of Diclofenac and Nanocin in 20% ethanol produced an opaque solution, which initially was thought to be due to insolubility, but it also occurred with a 30% ethanol vehicle and water.

When the combined formulation was processed through the Nanosight LM10 (nanoparticle detecting machine), the sample was too bright to read, but upon flushing the sample out, there were signs of many nanoparticles. The formulation had to be diluted 1 in 100 to get a level of nanoparticles that could be scanned. Even at this dilution, the number of particles was measured in the billions/ml (see FIG. 2a).

The data from the LM10 and also the DLS showed that the mean particle size for this formulation is approximately 150 nm, and the mode (from the LM10) is 138 nm. Ata 1:100 dilution of the 1:1 mg/ml solution the number of particles was 7×10⁹ particles/ml. The polydispersity index was described as good on the DLS. The population profile can be seen in FIG. 2b.

### EM Analysis of the Diclofenac Formulation

The formulation was also examined under scanning electron microscopy (SEM) by EM Support Systems Ltd, UK. First as a dry sample coated in gold and also using WET SEM.

The electron micrograph shown in FIG. 3 shows that the particles were between 100-300 nm after dehydration. There was bridging between the nanoparticles which occurred during the dehydration process but could also be partly caused by residue polymer that has not been formed into a nanoparticle.

The WETSEM imaging of the nanoparticles was successfully completed and images were obtained. FIG. 4 shows a backscatter image of the nanoparticles in solution. As expected, the contrast was very low as the nanoparticles consist of only polymer and no heavier elements that could give greater contrast, however individual particles can be seen. In addition, some aggregation or denser regions was observed, this may have been due to the presence of free polymer, which will also be charged and attracted to the surface of the capsule.

Formulations with the Other API's

When the other API's were formulated with Nanocin at either a concentration of 0.33:1 mg/ml or 1:1 mg/ml (API:Nanocin) there was evidence of nanoparticles being formed in both.

Table 3 below shows the DLS data for 0.33:1 mg/ml API:Nanocin.

TABLE 3

| 0.33:1 mg/ml | | |
|---|---|---|
| API:N | z-average | PDI |
| R | 68.44 | 0.01 |
| R:N | 148.6 | 0.226 |
| D | 55.88 | 0.251 |
| D:N | 123.5 | 0.324 |
| C | 65.39 | 0.229 |
| C:N | 286.9 | 0.615 |
| T | 41.54 | 0.332 |
| T:N | 47.77 | 0.496 |
| I | 194.1 | 0.289 |
| I:N | 113.5 | 0.235 |

Table 4 below shows DLS data for 1:1 mg/mi API:Nanocin

TABLE 4

| 1:1 mg/ml | | |
|---|---|---|
| API:N | z-average | PDI |
| D:N | 151.9 | 0.201 |
| D | 379.4 | 0.817 |
| R:N | 824.8 | 1 |
| R | 1001 | 1 |
| T:N | 117.9 | 0.065 |
| T | 277.1 | 1 |
| C:N | 258.5 | 0.112 |
| C | 231.8 | 0.205 |
| I:N | 65.12 | 0.158 |
| I | 311.9 | 0.34 |

The PDI's were high with Rapamycin and only the D:N formulation at 1:1 mg/ml gave a ‘good’report with this but not at the 0.33:1 mg/ml.

### Microbiology Testing

The API's formulated with Nanocin were tested on 2 bacterial strains associated with acne. Staphylococcus epidermidis (S. epidermidis) and the anaerobic Pseudomonas acnes (P. acnes). Two types of assay were performed: (i) a bacterial lawn assay where the formulation was pipetted onto a Whatman filter disc and then added to a dried lawn of the bacteria growing on agar; and also, (ii) a liquid minimum inhibitory concentration (MIC) assay, where the bacteria were grown in 96 well plates.

The bacterial lawn assay was much faster and easier to grow with the S. epidermidis as it was under aerobic conditions. The P. acnes growth on the agar plates was slower and difficult to capture on a camera (although possible to view by eye; see FIGS. 5c and 5d).

The lawn assay was not as sensitive as the liquid MIC assay at detecting inhibitory bacterial growth. The assay showed Nanocin preventing bacterial growth down to 1 mg/ml (FIG. 5a) (which is a much higher concentration than that observed with the liquid MIC).

However, the assay showed that when Nanocin was formulated with the API's at a 1:1 mg/ml ratio they did not affect the Nanocin inhibiting the bacterial growth (FIG. 2b).

As shown in FIG. 5a, the ‘halo’ around the 3,2 and 1 (mg/ml) discs indicates an inhibition of the bacterial growth caused by the Nanocin as the vehicle control does not show that affect.

As shown in FIG. 5b the halo's around all the discs are of a similar size to Nanocin alone, showing that the API's are not affecting the Nanocin inhibition of the S. epidermidis growth. Only vehicle has full bacterial growth around the disc. (D=Diclofenac; T=Tacrolimus; I=Ibuprofen; R=Rapamycin, C=Cyclosporine; N=Nanocin).

FIG. 5c shows the ‘halo’ of clear agar around the Nanocin alone disc (N; 1 mg/ml)) indicates inhibition of the bacterial growth, which is not seen where there is Diclofenac (1, 0.5, 0.33, 0.25, 0.2, 0.1 mg/ml) or vehicle (v, 20% ethanol) alone.

FIG. 5d shows the inhibition of the bacterial growth can be seen by the ‘halo’ encircling the filter discs, similar to the Nanocin alone circle (N; 1 mg/ml) which contained varying ratios of Diclofenac:Nanocin. There was no bacterial inhibition in the vehicle control (V).

### Liquid MIC Assay

The liquid MIC assay, as with the lawn assay, was much faster to grow (24 hours) with S. epidermidis than the P. acnes (minimum of 5 days in an anaerobic chamber). However, results from both were reliable.

With the S. epidermidis, Nanocin had a MIC of 0.32 ug/ml and when the API's (D-Diclofenac; T-Tacrolimus; 1-Ibuprofen; R-Rapamycin; C-Cyclosporine) were formulated with Nanocin this MIC did not alter (FIGS. 5e & 5f).

With the P. acnes, Nanocin had an MIC of 0.63 ug/ml, which was not altered when formulated with any of the API's. The API's alone did not demonstrate any anti-bacterial activity but profiled the vehicle effect on the bacterial growth (Tacrolimus (T) in FIG. 5h typically shows the API alone response).

As Diclofenac was showing interesting findings with the particle formation, a series of formulations with Diclofenac and Nanocin was tested on the liquid MIC assay (the results for S. epidermidis growth with Nanocin and Diclofenac are shown in FIGS. 5i-5o). All ratios of Nanocin:Diclofenac did not alter the Nanocin MIC with both bacterial strains. Diclofenac alone at all concentrations (50-500 ug/ml) did not exhibit any bacterial growth inhibition.

A further anaerobic bacteria associated with acne is Pseudomonas granulosum (P. granulosum) and this was tested alongside P. acnes against Nanocin to establish the Nanocin MIC (results shown in FIG. 5p) but was not tested beyond that with the API's. The MIC for Nanocin against P. granulosum was 0.08 ug/ml Nanocin.

Table 5 below shows the liquid MIC data (ug/ml).

TABLE 5

| | P. Acnes | S. epidermidis | P. granulosum |
|---|---|---|---|
| Nanocin | 0.32 | 0.64 | 0.08 |
| Nanocin & API's | 0.32 | 0.64 | — |

### Inflammation Assay

FIGS. 6-7b shows the results for an anti-inflammatory assay established using THP-1 cells (a human monocytic cell line). The release of cytokines (looking particularly at those associated with acne inflammation-TNF-α, IL-8, IL-1α) after stimulation with lipopolysaccharide (LPS) is a valid model system to test compounds for potential anti-inflammatory effects (Reference 1)

To standardise conditions for the assay, a LPS dose (0-1 ug/ml) was investigated on TNF-α, IL-8 and IL-1α response, over a time period from 2,4 and 24 hours. The results showed that IL-1α and IL-8 had their greatest expression after 24 hours, whereas TNF-α was an earlier responder between 2 and 4 hours, which is well known in the inflammatory cascade. As IL-8 and IL-1α both indicated a later stage in the inflammatory response and IL-8 showed a clearer response curve, IL-8 was progressed through into the screening process and IL-1α was discontinued.

API Dose Range on the Response with IL-8

A dose range of 0-30 ug/ml of each API was tested on the THP-1 cell assay (see FIG. 8a). Rapamycin, Diclofenac, Cyclosporine & Tacrolimus appeared more potent than Ibuprofen at inhibiting the LPS inflammatory response. Tacrolimus and Cyclosporine MIC was 0.3 ug/ml and Diclofenac & rapamycin at 1 ug/ml.

The API's were then tested with and without Nanocin formulation at a concentration of 30 ug/ml API and 100 ug/ml Nanocin. As previously shown Ibuprofen (I) had very little effect on reducing the IL-8 release, whereas the other API's did (FIG. 8b).

The results from FIG. 8b were normalised (FIG. 8c) with the cell count of each sample which identified Tacrolimus as a more effective anti-inflammatory at 30 ug/ml.

With the formulated samples and Nanocin alone the levels of IL-8 dropped nearly to zero (FIG. 8c) and this wasn't due to an inhibition of the IL-8 release but due to cell death (FIG. 8d) caused by Nanocin.

The THP-1 cells are sensitive to the Nanocin concentration so a Nanocin dose was performed on the cells (FIG. 8e). There was significant effect with Nanocin on cell viability at a concentration of 10 ug/ml after 2 hours and 1 ug/ml after 24 hours.

As shown in FIGS. 9-12, formulations were tested at a sufficiently low concentration of Nanocin that did not affect the cell viability. A 1:1 mg/ml (Nanocin:API) was initially formulated in 20% ethanol and the stock formulation was then diluted down with serum free media and tested at a final concentration of 1 or 0.1 ug/ml.

### Summary

Both cytokine measurements showed that Diclofenac alone and Nanocin alone reduced the LPS-stimulated inflammatory response. When formulated together, the levels of cytokine secreted was still considerably less than the LPS-stimulated response. The data suggests that Nanocin alone could have some anti-inflammatory action.

### Topical Skin Work

In vitro permeation studies were performed using porcine ear skin as described in greater detail in Example 2.

The experimental work involved applying various formulations to the skin in a Franz cell, leaving it for 24 hours as an infinite dose. Initial formulations were 1 mg/ml Nanocin and 300 ug/ml API in 20% ethanol. The same concentration of formulation were also used using FITC labelled Nanocin. Methods of detection was by: Franz Cell Method 1: Full OCT Embedding Method and Cryo-Sectioning; Franz Cell Method 2: Partial OCT Embedding and Cryo-Sectioning; Franz Cell Method 3: Partial OCT Embedding and Tape Stripping; and Time-of-Flight Secondary Ion Mass Spectrometry (ToF-SIMS) Fluorescence Microscopy.

In brief, at this concentration none of the API's were detectable in all the methods, apart from Diclofenac formulated with Nanocin, that had new secondary ions within the stratum corneum. It was hypothesised that the co-formulation of the diclofenac with the nanocin was causing substantive change in the ionisation pattern of the compound that resulted in a different fingerprint.

As diclofenac was showing signs of a signal it was decided to progress Tacrolimus as another class of anti-inflammatory agent, already used in skin remedies, ISAC was then provided with a higher dose of formulation as 1:1 mg/ml API:Nanocin to improve the signal in the ToF SIMS.

The ToF-SIMS analysis of the top 3 tape strips from 3 diclofenac and 3 diclofenac+nanocin appeared to suggest that the combination formulation induced permeation of the active ingredient into the top layers of the stratum corneum where the active alone does not.

Presented are distributions of CN— (marker for skin chemistry) and Cl—, NaCl₂— and Na₂Cl₃— which were used as markers for the diclofenac (salt). These were used based on a peak search looking for variance between the two sample types and a control (blank).

The CN— marker is used to showcase the successful stripping of skin tissue, and the respective localisation of this tissue on the tape strip. While Cl— was seen to be somewhat ubiquitous and is to a limited extent associated with native skin chemistry, the NaCl₂— and Na₂Cl₃— ion markers showed a strong variance compared to the control samples and do appear to correlate with the active ingredient. They are logical fragments of the salt structure of the compound.

Comparing the diclofenac+nanocin to the diclofenac alone samples it can be readily determined that there is a substantial, albeit heterogeneous presence of the NaCl₂— and Na₂Cl₃— ions in tape strips 1-3 of all the former samples, but none of the latter. Cl— is present in all the tape strips from both sample series, but shows a marked increase in intensity in the combination formulation.

Plots of the ion intensity data from all the samples and then combined into their respective groups supports this assertion.

One example of the Diclofenac & nanocin results is shown in FIG. 15. The ToF-SIMS cross sectional analysis comparing the 1 mg/ml Diclofenac and 1 mg/ml Diclofenac+Diclofenac+Nanocin could be seen to suggest that the nanocin formulation promoted (heterogeneously distributed) permeation into the stratum corneum, where there was reduced evidence of the same with the Diclofenac only formulation.

Sample preparation by the partial embedding method appeared to provide better sample stability (left with underlying cartilage) and reduced the impact of the OCT on image analysis.

CN— and PO2- were used as markers for the skin chemistry, while Cl—, NaCl2- and Na2Cl3- were used as markers for the Diclofenac (salt).

Notably, the control samples show no evidence of these markers accumulated in the stratum corneum. The diclofenac alone samples showed a slight elevation in the intensity of these ions in the stratum corneum region, and in the epidermis in general. However the diclofenac+nanocin samples show significant elevations in the stratum corneum, presenting as inconsistent, heterogeneous spikes in intensity. These often correlate with suppression of the PO2- signal that helps confirm the localisation.

### Example 2—In Vitro Permeation Assessment of Topically Delivered Active Pharmaceutical Ingredient with and without a Permeation Enhancer

### Background

The aims of these experiments were to assess the ‘in-vitro’ permeation of selected active pharmaceutical ingredients (APIs) on porcine skin sections, with and without Nanocin as a permeation enhancer.

During the experiments, it was planned that a Franz cell protocol was to be developed to model the topical delivery and subsequent permeation of Rapamycin, Tacrolimus, Ibuprofen, Ciclosporin and Diclofenac APIs. These APIs were then topically applied to porcine skin both alone and co-formulated Nanocin. The skin sections recovered from the Franz cells were then to be cryo-blocked and subsequently cryo-sectioned to provide cross sectional slices of tissue. The sections were then be chemically imaged by Time-of-Flight Secondary Ion Mass Spectrometry (ToF-SIMS) and fluorescence microscopy (FM) to localise the APIs and assess the extent to which they have permeated the skin sections. Secondary ion peaks representative of the APIs as pure materials were to be characterised in the initial phase of the project. These were to be used in the first instance to identify the API distributions. An (additional) FITC labelled nanocin variant was used to provide a fluorescence active permeation enhancer, which could then be detected by FM to determine permeation and tissue localisation of the range of formulations. The fluorescence work-up was performed in the first part of the project.

### Franz Cell Method 1: Full OCT Embedding Method and Cryo-Sectioning

Porcine ears used for the Franz cell analysis were sourced from a local abattoir. The age of the pig slaughtered were between 4-6 months old. The ears were cleaned with deionised water and the outside skin was carefully removed from the underlying cartilage. The excised skin was then stored at −20° C. until use. All ears used for the permeation experiment were within 6 months after procurement.

Prior to setting up the Franz cells, the skin was defrosted by leaving it at room temperature and pressure. Excess hairs on the porcine skin were not trimmed in this instance (to promote capacity to identify follicular delivery). The skin sections were directly cut to smaller section sizes with a diameter of 3 cm to ensure that the skin could be mounted in between the donor and receptor chamber of the Franz diffusion cells.

The receptor chambers were filled with 3 ml of 10% ethanol in phosphate buffer saline (PBS). Upon assembling the Franz cells, the skin was allowed to equilibrate in a 37° C. water bath for 30 minutes. This was carried out to ensure the skin reaches physiological temperature, 32° C. The skin was then treated with desired API formulations.

After 23 hours, the excess formulation was removed from the skin and cleaned with 3% Teepol solution using a non-scratching sponge. The skin sections were then cut into 1 cm×1 cm squares (corresponding to the effective area of the treated skin site). This skin section was then cut into half so that it can fit into a base mold containing optimal cutting temperature (OCT) resin. The skin sections were placed upright so that when sectioned, vertical cross-sections are obtained. The base molds were placed on a cooled aluminium block in a liquid nitrogen bath to allow the OCT to set. The molds were then stored at −80° C. until cross-sectioned. Sequential cross sectioning was then performed using a Leica CM 3050 S cryostat to generate a number of cross sectional slices for image analysis.

### Franz Cell Method 2: Partial OCT Embedding and Cryo-Sectioning

The porcine skin was sourced and underwent pre-preparation in the same fashion as in method 1 above. The ears were cleaned with deionised water and then stored at −20° C. until use. All ears used for the permeation experiment were within 6 months of procurement. For this experimental set up, inside skin attached to cartilage was used to generate sections with enhanced stability.

Prior to setting up the Franz cells, the skin was defrosted by leaving it at room temperature and pressure. Excess hairs on the porcine skin were again not trimmed as per a standard protocol (to promote capacity to identify follicular delivery) and the skin excepts were cut to smaller sections with a diameter of 3 cm for mounting in between the donor and receptor chamber of the Franz diffusion cells. The receptor chambers were filled with 3 ml of 10% ethanol in phosphate buffer saline (PBS).

Upon assembling the Franz cells, the skin was allowed to equilibrate in a 37° C. water bath for 30 minutes. This was carried out to ensure the skin reaches physiological temperature, 32° C. The skin was then treated with selected formulation. After 23 hours excess formulation was removed from the skin and the section washed with 3% Teepol solution using a non-scratching sponge.

The skin sections were cut into a 1 cm×1 cm square (corresponding to the treated skin site area). This reduced skin section was then cut in half placed on a cooled aluminium block in a liquid nitrogen bath to freeze the skin solid. These frozen skin sections were then placed upright in a base mould partially filled with OCT, with the goal of ensuring the portion of skin for sectioning is not embedded in OCT.

The skin sections were then stored at −80° C. until cross-sectioned. Cryo-sectioning was performed using a Leica CM 3050 S cryostat to a thickness of 20 μm. Resultant sections were transferred onto glass microscope slides and progressed to imaging analysis.

### Franz Cell Method 3: Partial OCT Embedding and Tape Stripping

Porcine skin was sourced and underwent standard preparation and Franz cell processing according to the same steps as laid out in method 1 (above) up to the removal of the samples from the Franz cells after treatment.

When the samples were cut down to 1 cm×1 cm following Franz cell extraction, they were subject to sequential tape stripping according to standardised protocol. Adhesive filmstrips were applied and removed successively to the treated skin area. The adhesive tape was pressed onto the skin using a roller to stretch the skin surface. 15 tape strip layers were collected for each sample prepared according to this method. Resultant tape strips were progressed to imaging analysis.

### Time-of-Flight Secondary Ion Mass Spectrometry (ToF-SIMS)

All ToF-SIMS sample section analysis was carried out on a ToF-SIMS IV instrument (ION-TOF GmbH., Munster, Germany) under ultra high vacuum conditions with operational parameters as below:
Primary Ion Beam: Bismuth liquid metal ion gun (Bi3+) 25 kV (pulsed target current of ˜1.0)
Sputter Ion Beam: NA
Analyser: Single-stage reflectron
Charge Compensation: pA. Low-energy electrons (20 eV)
Data acquisition and analysis: Was performed using SurfaceLab 6 software (IONTOF GmbH).

Details specific to Imaging:
Area analysed: Data was acquired over 500 μm×500 μm areas.
Resolution: 256×256 pixels
Scan no: 20 scans

### Fluorescence Microscopy

Sample sections for fluorescence microscopy were imaged using an inverted Nikon Eclipse T1 and QIMAGING optiMOS camera equipped with CoolLED pE-4000 fluorescence illumination, pE-100 bright field illumination and a Nikon plan Fluor 10× (0.30 NA) objective. Fluorescence was captured through excitation at 490 nm collecting emission at 410-500 (exposure time 50 μs), 500-550 (exposure time 200 μs), 550-650 (exposure time 200 μs) and 650-750 (exposure time 200 μs). Bright field was captured at an exposure time of 10 μs. All fluorescent and bright field images were corrected to a 12 bit image (0-4095).

FIG. 16 schematically shows the sample preparation for chemical imaging, whereas FIG. 17 shows the cross-sectional analysis vs tape strip analysis. FIG. 18 shows example section images (H&E stained).

### Experimental Plan

The experimental plan at the outset of the project was to prepare and analyse:

1. API+Nanocin Cross Sections for ToF-SIMS
Ciclosporin+Nanocin Cross Sections
Ibuprofen+Nanocin Cross Sections
Rapamycin+Nanocin Cross Sections
Tacrolimus+Nanocin Cross Sections
Diclofenac+Nanocin Cross Sections

2. API Cross Sections for ToF-SIMS
Ciclosporin Cross Sections
Ibuprofen Cross Sections
Rapamycin Cross Sections
Tacrolimus Cross Sections
Diclofenac Cross Sections

3. API+FITC-Nanocin Cross Sections for FM
Ciclosporin+FITC-Nanocin Cross Sections
Ibuprofen+FITC-Nanocin Cross Sections
Rapamycin+FITC-Nanocin Cross Sections
Tacrolimus+FITC-Nanocin Cross Sections
Diclofenac+FITC-Nanocin Cross Sections

### ToF-SIMS API+Nanocin Cross Sectional Analysis

Ciclosporin+Nanocin Samples all failed with stratum corneum delamination. Ibuprofen+Nanocin samples all failed with stratum corneum delamination. Rapamycin+Nanocin (2 Samples successfully prepared) Illustrative data shown in FIG. 19. There was no evidence of the rapamycin markers identified in active workup at skin surface, or within stratum corneum that may imply permeation. However, this may have been due to the sensitivity of the detection assay. Ions showing most spatial variance associated with the OCT and skin chemistry and were consistent with control sample (blank). Complete absence of (inability to detect) rapamycin suggested.

### Tacrolimus+Nanocin

One sample was successfully prepared and 1 sample rejected for heavy contamination. The data is shown in FIG. 20. No evidence of tacrolimus ion markers were identified in the API workup at the skin surface, or within stratum corneum layers that would imply permeation. Ions showing most variance again appear associated with skin chemistry and OCT medium and were consistent with control sample (blank). Complete absence of (or the inability to detect) tacrolimus suggested.

### Diclofenac+Nanocin

Two samples were successfully prepared and FIG. 21 shows the data. There was no evidence of the diclofenac ion markers identified in the API workup at the skin surface, or within stratum corneum layers that would imply permeation. However other ions (C14H27O2-, C16H31O2-, C14H29O8- and C22H43O2-), which were not seen in the reference work up can be seen to demonstrate a spatial variance consistent with localisation to the stratum corneum. These ions were seen to be in the mass range of (200-400 m/z) and were absent from control samples (untreated). This may suggest that the co-formulation of the nanocin-Diclofenac has sufficient impact on the ionization matrix of the compound chemistry as to produce a significantly different secondary ion fingerprint. If this is the case, then the ions seen in this analysis may reflect permeation of the diclofenac-nanocin complex, but this needs further work to expand.

The ToF-SIMS analysis of the API+nanocin cross section samples highlighted several key points:
Consistency of preparation of the porcine skin samples for this analysis was poor. Delamination of the stratum corneum was seen to be a persistent issue, identifying poor structural integrity of the samples undergoing these treatments. As such data was not collected for all APIs
No evidence could be gathered on the samples produced successfully for localisation of the APIs based on the secondary ion markers identified from the references.
However in the case of diclofenac, new secondary ions of interest were seen by a contrast search to associate with the stratum corneum.
It was hypothesised that the co-formulation of the diclofenac with the nanocin was causing a substantive change in the ionisation pattern of the compound that resulted in a different fingerprint.
If valid, then these new markers may suggest permeation of the diclofenac into the skin.
However it is also unclear if detection of the APIS/nanocin is reflective of a limit of detection.

### ToF-SIMS Tape Strip Analysis

The following experiments were used to maximise the analytical area the API/nanocin should be detectable and ensure lack of detection was not a threshold issue.
Ciclosporin+Nanocin Tape Strips
Ibuprofen+Nanocin Tape Strips
Rapamycin+Nanocin Tape Strips
Tacrolimus+Nanocin Tape Strips
Diclofenac+Nanocin Tape Strips

### ToF-SIMS—API+Nanocin Tape Strip Analysis

FIG. 22a shows the results for Ciclosporin+Nanocin. Ibuprofen+Nanocin samples all failed with stratum corneum delamination. FIG. 22b shows the results for Rapamycin+Nanocin. FIG. 22c shows the results for Tacrolimus+Nanocin. For Diclofenac+Nanocin samples all failed with stratum corneum delamination.

The ToF-SIMS analysis of the API-nanocin treated tape strip samples was consistent with the cross sectional data:
Consistency of preparation of the porcine skin samples for this analysis was again seen to be an issue. Delamination of the stratum corneum was persistent, identifying poor structural integrity of the samples undergoing these treatments. As such data was not collected for all APIs
The data collected for ciclosporin, rapamycin and tacrolimus formulations (with nanocin) showed that the ion intensity of the representative secondary ions determined in S1.1 was seen to be equivocal (in tape strips 1 and 2) to that seen in a control (blank sample).
This supported a lack of evidence for any permeation, and limited/no evidence of presence at the top surface at all.

### Fluorescence Microscopy of Tape Strips

Two API (Diclofenac and Tacrolimus) FITC-nanocin samples were chosen to assess whether FM imaging would showcase any obvious permeation in contradiction to the ToF-SIMS data.
Diclofenac+FITC-nanocin Tape Strips
Tacrolimus+FITC nanocin Tape Strips

The data for API+FITC-Nanocin Tape Strip Analysis is shown in FIGS. 23a-23c. FITC labelled nanocin-Diclofenac and Tacrolimus treated skin samples were generated using Franz cell method 3 (Partial OCT embedding with tape stripping) to support the investigation of the capacity to detect the API/nanocin formulations post treatment. A blank sample (no treatment) was also prepared to act as a control.

The tape stripped samples provide a lateral view of the skin surface which should maximise the capacity to detect the actives (fluorophore) relative the cross sectional preparation.

The top 3 tape strip layers (TS) from the stacks collected were imaged by FM to assess whether permeation of the nanocin-API complex could be inferred and assessed by the localisation of the fluorophore.

The illustrative images above (TS 1 and 2) and fluorescent intensity (FI) data collected suggested there was no significant different between intrinsic fluorescence seen on the control samples relative to the Tacrolimus and Diclofenac samples. The Diclofenac samples visually appeared to show more fluorescence on TS1 (top surface) but this was not identified as statistically significant by FI.

This data supported the ToF-SIMS tape strip data that there was no evidence of the critical components (APIs/nanocin) permeating or residing on the skin surface.

### Diclofenac vs Diclofenac+Nanocin Elevated Concentration ToF-SIMS Analysis

As Diclofenac was the only active ingredient where some suggestion of permeation could be identified (cross sectional analysis) it was decided to focus solely on this API, with and without nanocin. It was also determined that an elevated concentration of the API in the formulation (1 mg/ml) would be used to increase detection efficacy.

Furthermore to address sample preparation issues and improve the structural integrity of the skin sections (avoid delamination) an adjusted preparation method was used. Skin sections still attached to underlying cartilage were used to provide enough support to enable a partial embedding technique to be used. This provided a physical structure that was more robust, and also had the added benefit of reducing analytical issues around OCT leaching and complicating of image interpretation.

These experiments investigated the following:
Diclofenac+Nanocin Tape Strips
Diclofenac Tape Strips
Diclofenac+Nanocin Cross Sections
Diclofenac Cross Sections

FIG. 24 shows ToF-SIMS Diclofenac+Nanocin TS1 (Repeats 1-3); FIG. 25 shows ToF-SIMS Diclofenac+Nanocin TS2 (Repeats 1-3); FIG. 26 shows ToF-SIMS Diclofenac+Nanocin TS3 (Repeats 1-3); FIG. 27 shows ToF-SIMS Diclofenac TS1; FIG. 28 shows ToF-SIMS Diclofenac TS2; FIG. 29 shows ToF-SIMS Diclofenac TS3

FIGS. 13 and 14 show the ToF-SIMS Ion Intensity Comparison used in the Tape Strip Analysis experiments.

### Observations

The ToF-SIMS analysis of the top 3 tape strips from 3 diclofenac and 3 diclofenac+nanocin appear to suggest that the combination formulation induced permeation of the active ingredient into the top layers of the stratum corneum where the active alone does not.

Presented are distributions of CN— (marker for skin chemistry) and Cl—, NaCl₂— and Na₂Cl₃— which were used as markers for the diclofenac (salt). These were used based on a peak search looking for variance between the two sample types and a control (blank).

The CN— marker is sued to showcase the successful stripping of skin tissue, and the respective localisation of this tissue on the tape strip. While Cl— was seen to be somewhat ubiquitous and is to a limited extent associated with native skin chemistry, the NaCl₂— and Na₂Cl₃— ion markers showed a strong variance compared to the control samples and do appear to correlate with the active ingredient. They are logical fragments of the salt structure of the compound.

Comparing the diclofenac+nanocin to the diclofenac alone samples it can be readily determined that there is a substantial, albeit heterogeneous presence of the NaCl₂— and Na₂Cl₃— ions in tape strips 1-3 of all the former samples, but none of the latter. Cl— is present in all the tape strips from both sample series, but shows a marked increase in intensity in the combination formulation.

Plots of the ion intensity data from all the samples and then combined into their respective groups supports this assertion.

FIG. 30 shows the results of Diclofenac vs Diclofenac+Nanocin.

The ToF-SIMS cross sectional analysis comparing the 1 mg/ml Diclofenac and 1 mg/ml Diclofenac+Diclofenac+Nanocin could be seen to suggest that the nanocin formulation promoted (heterogeneously distributed) permeation into the stratum corneum, where there was reduced evidence of the same with the Diclofenac only formulation.

Sample preparation by the partial embedding method appeared to provide better sample stability (left with underlying cartilage) and reduced the impact of the OCT on image analysis.

CN— and PO2- were used as markers for the skin chemistry, while Cl—, NaCl2- and Na2Cl3- were used as markers for the Diclofenac (salt).

Notably, the control samples show no evidence of these markers accumulated in the stratum corneum. The diclofenac alone samples showed a slight elevation in the intensity of these ions in the stratum corneum region, and in the epidermis in general. However the diclofenac+nanocin samples show significant elevations in the stratum corneum, presenting as inconsistent, heterogeneous spikes in intensity. These often correlate with suppression of the PO2- signal that helps confirm the localisation.

The key findings where that there was no evidence of cyclosporine, ibuprofen, rapamycin or tacrolimus permeation with or without Nanocin, although it cannot be discounted that this is due to sensitivity of the methodology meaning that these APIs were not detected in the skin samples. There was some evidence of enhanced diclofenac permeation when co-formulated with nanocin by tape stripping analysis and ToF-SIMS imaging.

### Summary

A full range of API treated skin samples were successfully generated by a Franz cell experimentation method. However subsequent sample progression to cross sectional slices proved to be inconsistent with several sample failures, most commonly attributed to stratum corneum inflammation and delamination. Initial data collected using the ToF-SIMS on the (non FITC labelled) nanocin-API formulation treated samples provided no evidence that neither the APIs nor nanocin could be detected using the ion markers identified. Experiments investigating API alone ToF-SIMS & API+FITC-Nanocin FM were not conducted and a method adjustment was initiated based on this data to carry out some lateral analysis of the skin surface via tape stripping to see whether the ion markers could be detected when looking over a larger expected surface area.

A more advanced data analysis of the secondary ion dataset for the cross sectional slices was also undertaken. This work highlighted that the Diclofenac-Nanocin sample sections showed some evidence of unique (relative to blank reference samples) secondary ion localisation to the stratum corneum. These markers (mass range 200-400 m/z) were not consistent with the reference markers listed from the reference work up. No such evidence was found for the other API systems. This suggested that the co-formulation of the Nanocin with the APIs was generating a unique ionisation matrix that resulted in different secondary ion structures to the APIs and the nanocin alone.

Fluorescent microscopy imaging of FITC-labelled nanocin—API treated samples presented no evidence of the fluorophore within the first 3 tape strip layers of the skin. An additional method change was initiated based on this data to exclusively focus on Diclofenac and use a variant on the sample preparation mechanism to improve sample stability. Higher concentrations (1 mg/ml) of the Diclofenac were formulated to ensure the limit of detection was been exceeded. A partial embedding protocol, on skin sections still attached to cartilage was used to good effect to improve sample viability under processing and remove the impact of OCT on image analysis and component leaching. Sample viability was improved, with reduced sample loss, and chemical imaging capacity was improved by removing the impact of the OCT chemistry. Both cross sectional slices and tape strips were prepared with Diclofenac (alone) and Diclofenac plus nanocin treated samples.

Tape stripping analysis of repeats of these two systems showed a difference in the localisation of the same ions identified in the ToF-SIMS cross sectional analysis but also a more pronounced other ion markers that logically correspond to the diclofenac structure. The tape strip data suggests heterogeneous permeation of the API in the nanocin formulated variant, with none in the API alone system. This was largely based on the use of ions relating the Diclofenac salt (Cl—, NaCl₂—, Na₂Cl₃—). The cross sectional analysis supports this assertion, suggesting permeation of diclofenac (by the same markers listed above) when co-formulated with nanocin into the stratum corneum. The distribution of these ions in the stratum corneum is somewhat heterogeneous, with spikes in intensity localised to particular points.

### Example 3—Human Skin Studies

Human skin studies confirmed the enhanced drug delivery of an NSAID (diclofenac) into healthy human skin (see FIGS. 31 and 32). Human abdominal skin was ethically sourced from a healthy human donor. Triplicate skin disks were placed into static diffusion cells (Franz cells) with the epidermal face uppermost. Drug solutions (diclofenac alone or diclofenac formulated with polyhexanide to form nanoparticles) were added to the upper chamber of the Franz cell. The Franz cells were fully assembled and then incubated at 32° C. for 24 hours before analysis. At this time point, the Franz cells were disassembled, and the skin disks removed. The disks were washed and dried by dabbing with a tissue. The upper layers of skin were then sequentially stripped off three times using adhesive tape. Samples were also taken from the upper chamber and lower chambers of the Franz cells.

All samples were analysed for the presence of diclofenac by quantitative LC-MS using a Waters ACQUITY QDa mass detector (FIG. 31). Additionally, samples from the tape strips were analysed for their ability to inhibit cyclooxygenase-1 (Cox-1) using an in vitro assay purchased from Abcam (FIG. 32).

At 24 hours, there was little detectable diclofenac in any of the receptor fluid samples showing that minimal drug had passed through the skin in this time. The only exception was for one of the diclofenac/polyhexanide samples where substantial amounts of the applied drug were found in the receptor fluid. However, this was due to leakage of the fluid past the skin disk in this one sample (FIG. 31; DN1:1_1).

In comparison to the diclofenac alone treated disks, the diclofenac/polyhexanide treated samples demonstrated significantly enhanced drug delivery into the upper layers of the skin as demonstrated by higher drug concentrations from diclofenac/polyhexanide skin tape strips compared to diclofenac alone treated skin (FIG. 31). As summarised Table 6 below, the ratio of drug between diclofenac/polyexanide:diclofenac alone treated samples in the individual tape strips increased with each sequential tape strip indicating not only enhanced drug association with the upper layers of the skin but enhanced penetration into the skin. The Cox-1 assay confirmed these observations and further demonstrated that the levels of diclofenac within the tape strips from the diclofenac/polyhexanide treated samples is sufficient to produce significant Cox-1 inhibition whereas the levels in the diclofenac alone treated samples did not (FIG. 32). Analysis of the amount of drug remaining in the upper chamber (FIG. 31) also demonstrated that in the diclofenac/polyhexanide solutions, the majority of drug had been lost from the chamber, presumably due to penetration into the skin. In contrast the majority of the applied drug remained in the upper chambers of the diclofenac alone treatments.

TABLE 6
Analysis of diclofenac concentration in
sequential tape strips of human skin

| | Concentration of diclofenac in tape strips for | | |
|---|---|---|---|
| | the indicated treatments (ug/ml) | | |
| Sequential | Diclofenac/ | Diclofenac | Ratio of drug |
| Tape | Nanocin | alone | in the tape |
| Strip | (D/N) | (D) | strips D/N:D |
| Tape Strip 1 | 10.94 | 0.42 | 25.8 |
| Tape Strip 2 | 6.39 | 0.24 | 26.6 |
| Tape Strip 3 | 5.79 | 0.18 | 32.1 |

samples treated with the indicated formulations

Following striping of the skin, tape strips were suspended in 5 ml of methanol to solubilize drug off the tape prior to analysis by LC-MS.

These results demonstrate clear, enhanced skin delivery of diclofenac into human skin following formulation of diclofenac with polyhexanide.

The forgoing embodiments are not intended to limit the scope of the protection afforded by the claims, but rather to describe examples of how the invention may be put into practice.

### REFERENCES

1. ‘Development of an in vitro screening assay to test the anti-inflammatory properties of dietary supplements and pharmacologic agents.’ Clinical Chemistry 51:12, 2252-2256 (2005) Uma Singh et al.

1. A composition for use in the treatment of a dermatological condition, comprising a polymer capable of forming nanoparticles and an anti-inflammatory and/or analgesic agent.

2. The composition as claimed in claim 1, wherein the polymer comprises a linear and/or branched or cyclic polymonoguanide/polyguanidine, polybiguanide, analogue or derivative thereof.

3. The composition as claimed in either claim 1 or 2, wherein the polymer comprises polyhexamethylene biguanide.

4. The composition as claimed in any preceding claim, wherein the nanoparticles are formed with and/or in the presence of the anti-inflammatory and/or analgesic agent.

5. The composition as claimed in any preceding claim, wherein the anti-inflammatory agent comprises a non-steroidal anti-inflammatory (NSAID) agent.

6. The composition as claimed in any preceding claim, wherein the anti-inflammatory agent comprises one or more selected from the following: Rapamycin, Tacrolimus, Ibuprofen, Ciclosporin, Diclofenac, Naproxen and related derivatives and salts thereof.

7. The composition as claimed in any preceding claim, wherein the composition further comprises one or more of the following component: buffers, excipients, binders, oils, water, emulsifiers, glycerin, antioxidants, preservatives and fragrances.

8. The composition as claimed in any preceding claim, wherein the composition is in the form of a topical medicament.

9. The composition as claimed in any preceding claim, wherein the dermatological condition comprises acne and/or atopic dermatitis or related conditions.

10. A composition as claimed in any preceding claim, wherein the composition is in the form of a cream, ointment, spray, powder, foam or mousse.

11. Use of polyhexamethylene biguanide (PHMB) to form one or more nanoparticles with, or associated with, an anti-inflammatory and/or analgesic agent in the preparation of a medicament for the treatment of a dermatological condition.

12. Use of PHMB as claimed in claim 11, wherein the nanoparticles are used as the delivery vehicle for anti-inflammatory and/or analgesic agent to an affected dermatological area.

13. Use of PHMB as claimed in claim 11 or 12, wherein the dermatological condition comprises acne and/or atopic dermatitis or related conditions.

14. Use of PHMB as claimed in any one of claims 11 to 13, wherein the anti-inflammatory and/or analgesic agent comprises a non-steroidal anti-inflammatory (NSAID) agent.

15. Use of PHMB as claimed in any one of claims 11 to 14, wherein the anti-inflammatory and/or analgesic agent comprises one or more selected from the following: Rapamycin, Tacrolimus, Ibuprofen, Ciclosporin, Diclofenac, Naproxen and related derivatives and salts thereof.

16. A method of producing a composition for the treatment of a dermatological condition comprising mixing a polymer capable of forming nanoparticles with an anti-inflammatory and/or analgesic agent under conditions suitable to allow the formation of nanoparticles.

17. The method as claimed in claim 16, wherein the method is used to produce a composition as claimed in any one of claims 1 to 10.
